(19) United States
(12) Patent Application Publication
(10) Pub. No.: US 2023/0372012 A1
(43) Pub. Date: Nov. 23, 2023

(54) DETECTING FAILURE MITIGATION ASSOCIATED WITH AUTONOMOUS SURGICAL TASK

(71) Applicant: Cilag GmbH International, Zug (CH)

(72) Inventors: Jason L. Harris, Lebanon, OH (US); Frederick E. Shelton, IV, Hillsboro, OH (US); Kevin M. Fiebig, Cincinnati, OH (US); Matthew David Cowperthwait, Cincinnati, OH (US); Curtis Anthony Maples, Cincinnati, OH (US); Shane R. Adams, Lebanon, OH (US); Nicholas James Ross, Franklin, OH (US); Raymond Edward Parfett, Loveland, OH (US)

(21) Appl. No.: 17/747,714

(22) Filed: May 18, 2022

(51) Int. Cl. A61B 34/10

(52) U.S. Cl. CPC A61B 34/10; A61B 34/32

(57) ABSTRACT

Systems, methods, and instrumentalities are described herein for detecting failure mitigation associated with a surgical task. A device may perform a first autonomous function associated with a surgical task. Performing the first autonomous function may be associated with control feedback. The first autonomous function may be monitored by tracking one or more outputs associated with the control feedback. If the one or more outputs cross a failure threshold, the device may switch from performing the first autonomous function to performing a second autonomous function associated with the surgical task. The second autonomous function may be associated with failure mitigation feedback.

### CROSS-REFERENCE TO RELATED APPLICATIONS

This application is related to the following, filed contemporaneously, the contents of each of which are incorporated by reference herein:
U.S. Patent Application, entitled METHOD OF CONTROLLING AUTONOMOUS OPERATIONS IN A SURGICAL SYSTEM, with attorney docket number END9430USNP1.
U.S. Patent Application, entitled DYNAMICALLY DETERMINING SURGICAL AUTONOMY LEVEL, with attorney docket number END9430USNP2.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and unproved technologies due to patient safety and a general desire for maintaining traditional practices.

### SUMMARY

Systems, methods, and instrumentalities are described herein for detecting failure mitigation associated with a surgical task. A device may perform a first autonomous function associated with a surgical task. Performing the first autonomous function may be associated with control feedback. The first autonomous function may be monitored by tracking one or more outputs associated with the control feedback. If the one or more outputs cross a failure threshold, the device may switch from performing the first autonomous function to performing a second autonomous function associated with the surgical task. The second autonomous function may be associated with failure mitigation feedback. The device may generate a failure magnitude based on the one or more outputs that crossed the failure threshold. The device may adjust one or more parameters associated with the failure mitigation feedback based on the failure magnitude. In examples, the failure magnitude may be generated based on one or more of the following: surgical context, type of the first autonomous function, type of the second autonomous function, historical data associated with the first autonomous function, a number of outputs that crossed failure threshold, or a degree of the one or more outputs crossing the failure threshold.

### BRIEF DESCRIPTION OF THE DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 illustrates a surgical data network having a set of communication surgical hubs configured to connect with a set of sensing systems, an environmental sensing system, a set of devices, etc.

FIG. 5 illustrates a logic diagram of a control system of a surgical instrument.

FIG. 6 shows an example surgical system that includes a handle having a controller and a motor, an adapter releasably coupled to the handle, and a loading unit releasably coupled to the adapter.

FIG. 7 shows an example situationally aware surgical system.

FIG. 8 shows an example of a surgical autonomous system.

FIG. 9 shows the relationship between control feedback and failure mitigation feedback.

FIG. 10 shows an example of performing autonomous function with an deal model and a failure model

FIG. 11 shows an example of performing an autonomous function with control feedback and failure mitigation feedback.

FIG. 12 shows an example of the overview of the surgical instrument with control feedback and the failure mitigation feedback and the surgical hub.

### DETAILED DESCRIPTION

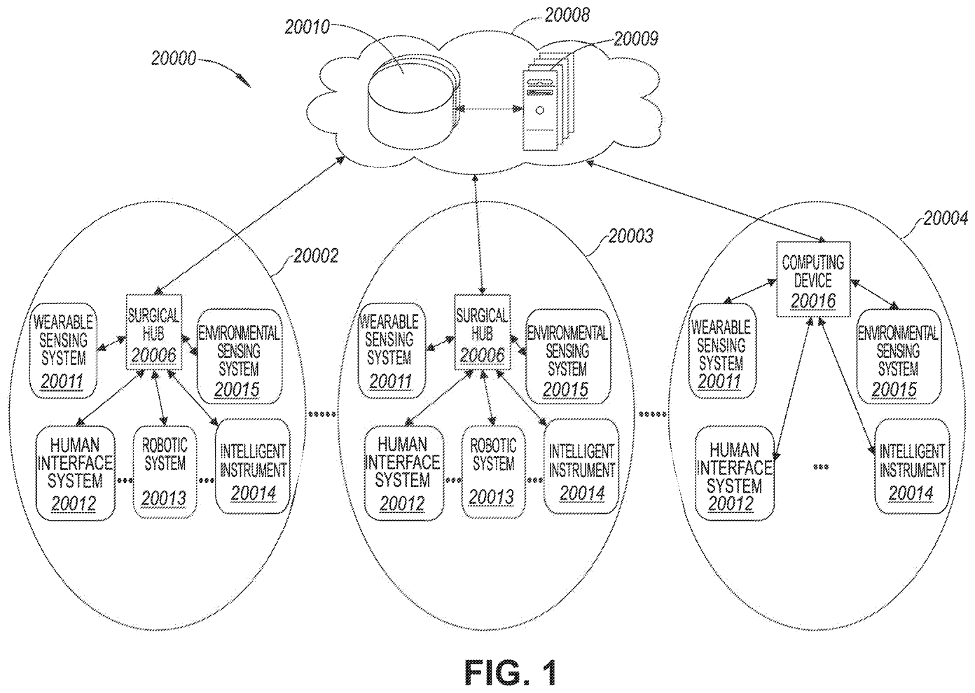

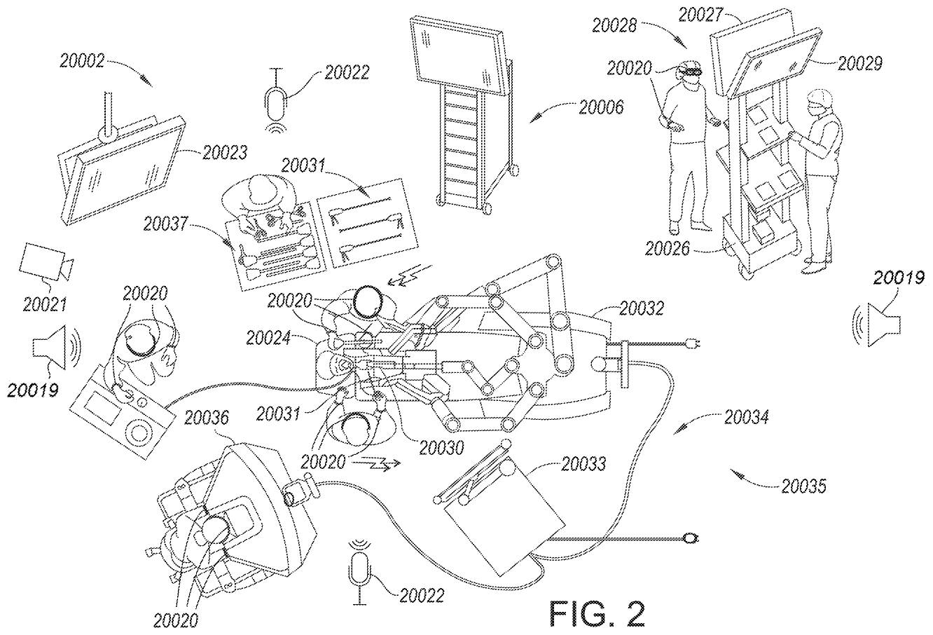

FIG. 1 is a block diagram of a computer-implemented surgical system 20000. An example surgical system such as the surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include a wearable sensing system 20011, an environmental sensing system 20015, a robotic system 20013, one or more intelligent instruments 20014, human interface system 20012, etc. The human interface system is also referred herein as the human interface device. The wearable sensing system 20011 may include one or more HCP sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G.

A surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical huh 20006 may be configured to gather measurement data from the one or more sensing systems 20011 and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems 20001 may include the wearable sensing system 20011 (which may include one or more HCP sensing systems and one or more patient sensing systems) and the environmental sensing system 20015 as discussed in FIG. 1. The one or more sensing systems 20001 may measure data relating to various biomarkers. The one or more sensing systems 20001 may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The one or more sensors may measure the biomarkers as described herein using one of more of the following sensing technologies: photoplethysmography, electrocardiography, electroencephalography, colorimetry, impedimentary, potentiometry, amperometry, etc.

The biomarkers measured by the one or more sensing systems 20001 may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example. The one or more sensing systems 20001, biomarkers 20005, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

FIG. 2 shows an example of a surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

In one aspect, the surgical hub 20006 may be configured to route a diagnostic input or feedback entered by a non-sterile operator at the visualization tower 20026 to the primary display 20023 within the sterile field, where it can be viewed by a sterile operator at the operating table. In one example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

FIG. 2 illustrates an example of a surgical system 20002 being used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (i.e., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more sensing systems, for example, HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, a sensing system 20020 may measure a set of biomarkers to monitor the heart rate of an HCP. In an example, a sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing. One or more environmental sensing devices may send environmental in to the surgical hub 20006. For example, the environmental sensing devices may include a camera 20021 for detecting hand/body position of an HCP. The environmental sensing devices may include microphones 20022 for measuring the ambient noise in the surgical theater. Other environmental sensing devices may include devices, for example, a thermometer to measure temperature and a hygrometer to measure humidity of the surroundings in the surgical theater, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors. In an example, the HCP sensing systems 20020 may measure one or more surgeon biomarkers associated with an HCP and send the measurement data associated with the surgeon biomarkers to the surgical hub 20006. The HCP sensing systems 20020 may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

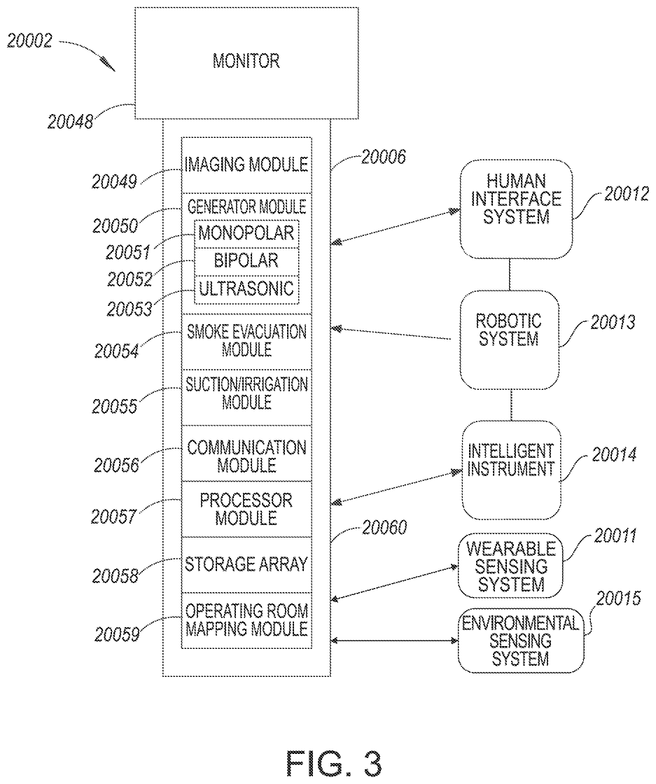

FIG. 3 shows an example surgical system 20002 with a surgical hub 20006. The surgical hub 20006 may, be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050, a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 20060 offers a unified environment for managing the power, data, and fluid lines, which reduces the frequency of entanglement between such lines. Aspects of the present disclosure present a surgical hub 20006 for use in a surgical procedure that involves energy application to tissue at a surgical site. The surgical hub 20006 includes a hub enclosure 20060 and a combo generator module slidably receivable in a docking station of the hub enclosure 20060. The docking station includes data and power contacts. The combo generator module includes two or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. In one aspect, the fluid line may be a first fluid line, and a second fluid line may extend from the remote surgical site to a suction and irrigation module 20055 slidably received in the hub enclosure 20060. In one aspect, the hub enclosure 20060 may include a fluid interface. Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. One of the advantages of the hub modular enclosure 20060 is enabling the quick removal and/or replacement of various modules. Aspects of the present disclosure present a modular surgical enclosure for use in a surgical procedure that involves energy application to tissue. The modular surgical enclosure includes a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. Further to the above, the modular surgical enclosure also includes a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module. Referring to FIG. 3, aspects of the present disclosure are presented for a hub modular enclosure 20060 that allows the modular integration of a generator module 20050, a smoke evacuation module 20054, and a suction/irrigation module 20055. The hub modular enclosure 20060 further facilitates interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 can be configured to connect to a monopolar device 20051, a bipolar device 20052, and an ultrasonic device 20053. Alternatively, the generator module 20050 may comprise a series of monopolar, bipolar, and/or ultrasonic generator modules that interact through the hub modular enclosure 20060. The hub modular enclosure 20060 can be configured to facilitate the insertion of multiple generators and interactive communication between the generators docked into the hub modular enclosure 20060 so that the generators would act as a single generator.

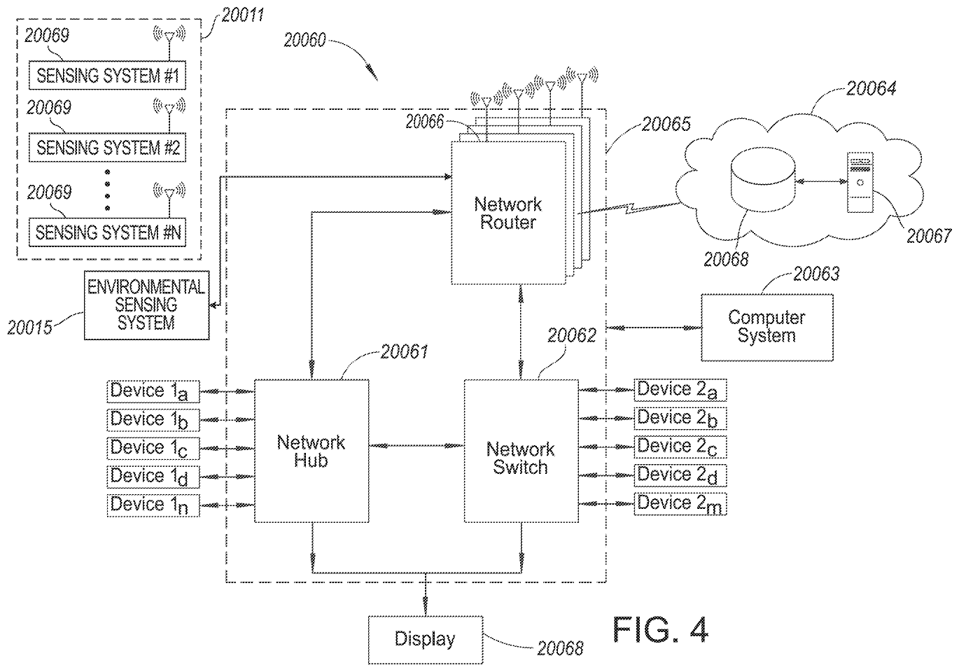

FIG. 4 illustrates a surgical data network having a set of communication hubs configured to connect a set of sensing systems, environment sensing system(s), and a set of other modular devices located in one or more operating theaters of a healthcare facility, a patient recovery room, or a room in a healthcare facility specially equipped for surgical operations, to the cloud, in accordance with at least one aspect of the present disclosure.

As illustrated in FIG. 4, a surgical hub system 20060 may include a modular communication hub 20065 that is configured to connect modular devices located in a healthcare facility to a cloud-based system (e.g., a cloud computing system 20064 that may include a remote server 20067 coupled to a remote storage 20068). The modular communication hub 20065 and the devices may be connected in a room in a healthcare facility specially equipped for surgical operations. In one aspect, the modular communication hub 20065 may include a network hub 20061 and/or a network switch 20062 in communication with a network router 20066. The modular communication hub 20065 may be coupled to a local computer system 20063 to provide local computer processing and data manipulation.

the computer system 20063 may comprise a processor and a network interface 20100. The processor may be coupled to a communication module, storage, memory, non-volatile memory, and input/output (I/O) interface via a system bus. The system bus can be any of several types of bus structure(s) including the memory bus or memory controller, a peripheral bus or external bus, and/or a local bus using any variety of available bus architectures including, but not limited to, 9-bit bus, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), USB, Advanced Graphics Port (AGP), Personal Computer Memory Card International Association bus (PCMCIA), Small Computer Systems Interface (SCSI), or any other proprietary bus.

The processor may be any single-core or multicore processor such as those known under the trade name ARM Cortex by Texas instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with StellarisWare® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

In an example, the processor may comprise a safety controller comprising two controller-based families such as TMS570 and RM4-x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

It is to be appreciated that the computer system 20063 may include software that acts as an intermediary between users and the basic computer resources described in a suitable operating environment. Such software may include an operating system. The operating system, which can be stored on the disk storage, may act to control and allocate resources of the computer system. System applications may take advantage of the management of resources by the operating system through program modules and program data stored either in the system memory or on the disk storage. It is to be appreciated that various components described herein can be implemented with various operating systems or combinations of operating systems.

A user may enter commands or information into the computer system 20063 through input device(s) coupled to the I/O interface. The input devices may include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processor 20102 through the system bus via interface port(s). The interface port(s) include, for example, a serial port, a parallel port, a game port, and a USB. The output device(s) use some of the same types of ports as input device(s). Thus, for example, a USB port may be used to provide input to the computer system 20063 and to output information from the computer system 20063 to an output device. An output adapter may be provided to illustrate that there can be some output devices like monitors, displays, speakers, and printers, among other output devices that may require special adapters. The output adapters may include, by way of illustration and not limitation, video and sound cards that provide a means of connection between the output device and the system bus. It should be noted that other devices and/or systems of devices, such as remote computer(s), may provide both input and output capabilities.

The computer system 20063 can operate in a networked environment using logical connections to one or more remote computers, such as cloud computer(s), or local computers. The remote cloud computer(s) can be a personal computer, server, router, network PC, workstation, microprocessor-based appliance, peer device, or other common network node, and the like, and typically includes many or all of the elements described relative to the computer system. For purposes of brevity, only a memory storage device is illustrated with the remote computer(s). The remote computer(s) may be logically connected to the computer system through a network interface and then physically connected via a communication connection. The network interface may encompass communication networks such as local area networks (LANs) and wide area networks (WANs). LAN technologies may include Fiber Distributed Data Interface (FDDI), Copper Distributed Data Interface (CDDI), Ethernet/IEEE 802.3, Token Ring/IEEE 802.5, and the like. WAN technologies may include, bat are not limited to, point-to-point links, circuit-switching networks like Integrated Services Digital Networks (ISDN) and variations thereon, packet-switching networks, and Digital Subscriber Lines (DSL).

In various examples, the computer system 20063 may comprise an image processor, image-processing engine, media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image-processing engine can perform a range of tasks. The image processor may be a system on a chip with multicore processor architecture.

The communication connection(s) may refer to the hardware/software employed to connect the network interface to the bus. While the communication connection is shown for illustrative clarity inside the computer system 20063, it can also be external to the computer system 20063. The hardware/software necessary for connection to the network interface may include, for illustrative purposes only, internal and external technologies such as modems, including regular telephone-grade modems, cable modems, optical fiber modems, and DSL modems, ISDN adapters, and Ethernet cards. In some examples, the network interface may also be provided using an RF interface.

Surgical data network associated with the surgical hub system 20060 may be configured as passive, intelligent, or switching. A passive surgical data network serves as a conduit for the data, enabling it to go from one device (or segment) to another and to the cloud computing resources. An intelligent surgical data network includes additional features to enable the traffic passing through the surgical data network to be monitored and to configure each port in the network hub 20061 or network switch 20062. An intelligent surgical data network may be referred to as a manageable hub or switch. A switching hub reads the destination address of each packet and then forwards the packet to the correct port.

Modular devices 1a-1n located in the operating theater may be coupled to the modular communication hub 20065. The network hub 20061 and/or the network switch 20062 may be coupled to a network router 20066 to connect the devices 1a-1n to the cloud computing system 20064 or the local computer system 20063. Data associated with the devices 1a-1n may be transferred to cloud-based computers via the router for remote data processing and manipulation. Data associated with the devices 1a-1n may also be transferred to the local computer system 20063 for local data processing and manipulation. Modular devices 2a-2m located in the same operating theater also may be coupled to a network switch 20062. The network switch 20062 may be coupled to the network hub 20061 and/or the network router 20066 to connect the devices 2a-2m to the cloud 20064. Data associated with the devices 2a-2m may be transferred to the cloud computing system 20064 via the network router 20066 for data processing and manipulation. Data associated with the devices 2a-2m may also be transferred to the local computer system 20063 for local data processing and manipulation.

The wearable sensing system 20011 may include one or more sensing systems 20069. The sensing systems 20069 may include an HCP sensing system and/or a patient sensing system. The one or more sensing systems 20069 may be in communication with the computer system 20063 of a surgical hub system 20060 or the cloud server 20067 directly via one of the network routers 20066 or via a network hub 20061 or network switching 20062 that is in communication with the network routers 20066.

The sensing systems 20069 may be coupled to the network router 20066 to connect to the sensing systems 20069 to the local computer system 20063 and/or the cloud computing system 20064. Data associated with the sensing systems 20069 may be transferred to the cloud computing system 20064 via the network router 20066 for data processing and manipulation. Data associated with the sensing systems 20069 may also be transferred to the local computer system 20063 for local data processing and manipulation.

As illustrated in FIG. 4, the surgical huh system 20060 may be expanded by interconnecting multiple network hubs 20061 and/or multiple network switches 20062 with multiple network routers 20066. The modular communication hub 20065 may be contained in a modular control tower configured to receive multiple devices 1a-1n/2a-2m. The local computer system 20063 also may be contained in a modular control tower. The modular communication hub 20065 may be connected to a display 20068 to display images obtained by some of the devices 1a-1n/2a-2m, for example during surgical procedures. In various aspects, the devices 1a-1n/2a-2m may include, for example, various modules such as an imaging module coupled to an endoscope, a generator module coupled to an energy-based surgical device, a smoke evacuation module, a suction/irrigation module, a communication module, a processor module, a storage array, a surgical device coupled to a display, and/or a non-contact sensor module, among other modular devices that may be connected to the modular communication hub 20065 of the surgical data network.

In one aspect, the surgical hub system 20060 illustrated in FIG. 4 may comprise a combination of network hub(s), network switch(es), and network router(s) connecting the devices 1a-1n/2a-2m or the sensing systems 20069 to the cloud-base system 20064. One or more of the devices 1a-1n/2a-2m or the sensing systems 20069 coupled to the network hub 20061 or network switch 20062 may collect data in real-time and transfer the data to cloud computers for data processing and manipulation. It will be appreciated that cloud computing relies on sharing computing resources rather than having local servers or personal devices to handle software applications. The word “cloud” may be used as a metaphor for “the Internet,” although the term is not limited as such. Accordingly, the term “cloud computing” may be used herein to refer to “a type of Internet-based computing,” where different services—such as servers, storage, and applications—are delivered to the modular communication hub 20065 and/or computer system 20063 located in the surgical theater (e.g., a fixed, mobile, temporary, or field operating room or space) and to devices connected to the modular communication hub 20065 and/or computer system 20063 through the Internet. The cloud infrastructure may be maintained by a cloud service provider. In this context, the cloud service provider may be the entity that coordinates the usage and control of the devices 1a-1n/2a-2m located in one or more operating theaters. The cloud computing services can perform a large number of calculations based on the data gathered by smart surgical instruments, robots, sensing systems, and other computerized devices located in the operating theater. The hub hardware enables multiple devices, sensing systems, and/or connections to be connected to a computer that communicates with the cloud computing resources and storage.

Applying cloud computer data processing techniques on the data collected by the devices 1a-1n/2a-2m, the surgical data network can provide improved surgical outcomes, reduced costs, and improved patient satisfaction. At least some of the devices 1a-1n/2a-2m may be employed to view tissue states to assess leaks or perfusion or sealed tissue after a tissue sealing and cutting procedure. At least some of the devices 1a-1n/2a-2m may be employed to identify pathology, such as the effects of diseases, using the cloud-based computing to examine data including images of samples of body tissue for diagnostic purposes. This may include localization and margin confirmation of tissue and phenotypes. At least some of the devices 1a-1n/2a-2m may be employed to identify anatomical structures of the body using a variety of sensors integrated with imaging devices and techniques such as overlaying images captured by multiple imaging devices. The data gathered by the devices 1a-1n/2a-2m, including image data, may be transferred to the cloud computing system 20064 or the local computer system 20063 or both for data processing and manipulation including image processing and manipulation. The data may be analyzed to improve surgical procedure outcomes by determining if further treatment, such as the application of endoscopic intervention, emerging technologies, a targeted radiation, targeted intervention, and precise robotics to tissue-specific sites and conditions, may be pursued. Such data analysis may further employ outcome analytics processing and using standardized approaches may provide beneficial feedback to either confirm surgical treatments and the behavior of the surgeon or suggest modifications to surgical treatments and the behavior of the surgeon.

Applying cloud computer data processing techniques on the measurement data collected by the sensing systems 20069, the surgical data network can provide improved surgical outcomes, improved recovery outcomes, reduced costs, and improved patient satisfaction. At least some of the sensing systems 20069 may be employed to assess physiological conditions of a surgeon operating on a patient or a patient being prepared for a surgical procedure or a patient recovering after a surgical procedure. The cloud-based computing system 20064 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The operating theater devices 1a-1n may be connected to the modular communication hub 20065 over a wired channel or a wireless channel depending on the configuration of the devices 1a-1n to a network hub 20061. The network hub 20061 may be implemented, in one aspect, as a local network broadcast device that works on the physical layer of the Open System Interconnection (OSI) model. The network hub may provide connectivity to the devices 1a/1n located in the same operating theater network. The network hub 20061 may collect data in the form of packets and sends them to the router in half duplex mode. The network hub 20061 may not store any media access control/Internet Protocol (MAC/IP) to transfer the device data. Only one of the devices 1a-1n can send data at a time through the network hub 20061. The network hub 20061 may not have routing tables or intelligence regarding where to send information and broadcasts all network data across each connection and to a remote server 20067 of the cloud computing system 20064. The network hub 20061 can detect basic network errors such as collisions but having all information broadcast to multiple ports can be a security risk and cause bottlenecks.

The operating theater devices 2a-2m may be connected to a network switch 20062 over a wired channel or a wireless channel. The network switch 20062 works in the data link layer of the OSI model. The network switch 20062 may be a multicast device for connecting the devices 2a-2m located in the same operating theater to the network. The network switch 20062 may send data in the form of frames to the network router 20066 and may work in full duplex mode. Multiple devices 2a-2m can send data at the same time through the network switch 20062. The network switch 20062 stores and uses MAC addresses of the devices 2a-2m to transfer data.

The network hub 20061 and/or the network switch 20062 may be coupled to the network router 20066 for connection to the cloud computing system 20064. The network router 20066 works in the network layer of the OSI model. The network router 20066 creates a route for transmitting data packets received from the network hub 20061 and/or network switch 20062 to cloud-based computer resources for further processing and manipulation of the data collected by any one of or all the devices 1a-1n/2a-2m and wearable sensing system 20011. The network router 20066 may be employed to connect two or more different networks located in different locations, such as, for example, different operating theaters of the same healthcare facility or different networks located in different operating theaters of different healthcare facilities. The network router 20066 may send data in the form of packets to the cloud computing system 20064 and works in full duplex mode. Multiple devices can send data at the same time. The network router 20066 may use IP addresses to transfer data.

In an example, the network hub 20061 may be implemented as a USB hub, which allows multiple USB devices to be connected to a host computer. The USB hub may expand a single USE port into several tiers so that there are more ports available to connect devices to the host system computer. The network hub 20061 may include wired or wireless capabilities to receive information over a wired channel or a wireless channel. In one aspect, a wireless USB short-range, high-bandwidth wireless radio communication protocol may be employed for communication between the devices 1a-1n and devices 2a-2m located in the operating theater.

In examples, the operating theater devices 1a-1n/2a-2m and/or the sensing systems 20069 may communicate to the modular communication hub 20065 via Bluetooth wireless technology standard for exchanging data over short distances (using short-wavelength UHF radio waves in the ISM band from 2.4 to 2.485 GHz) from fixed and mobile devices and building personal area networks (PANs). The operating theater devices 1a-1n/2a-2m and/or the sensing systems 20069 may communicate to the modular communication hub 20065 via a number of wireless or wired communication standards or protocols, including but not limited to Bluetooth, Low-Energy Bluetooth, near-field communication (NFC), Wi-Fi (IEEE 802.11 family), WiMAX (IEEE 802.16 family), IEEE 802.20, new radio (NR), long-term evolution (LTE), and Ev-DO, HSPA+, HSDPA+, HSUPA+, EDGE, GSM, GPRS, CDMA, TDMA, DECT, and Ethernet derivatives thereof, as well as any other wireless and wired protocols that are designated as 3G, 4G, 5G, and beyond. The computing module may include a plurality of communication modules. For instance, a first communication module may be dedicated to shorter-range wireless communications such as Wi-Fi and Bluetooth Low-Energy Bluetooth, Bluetooth Smart, and a second communication module may be dedicated to longer-range wireless communications such as GPS, EDGE, GPRS, CDMA, WiMAX, LTE, Ev-DO, HSPA+, HSDPA+, HSUPA+, EDGE, GSM, GPRS, CDMA, TDMA, and others.

The modular communication hub 20065 may serve as a central connection for one or more of the operating theater devices 1a-1n/2a-2m and/or the sensing systems 20069 and may handle a data type known as frames. Frames may carry the data generated by the devices 1a-1n/2a-2m and the sensing systems 20069. When a frame is received by the modular communication hub 20065, it may be amplified and/or sent to the network router 20066, which may transfer the data to the cloud computing system 20064 or the local computer system 20063 by using a number of wireless or wired communication standards or protocols, as described herein.

The modular communication hub 20065 can be used as a standalone device or be connected to compatible network hubs 20061 and network switches 20062 to form a larger network. The modular communication hub 20065 can be generally easy to install, configure, and maintain, making it a good option for networking the operating theater devices 1a-1n/2a-2m.

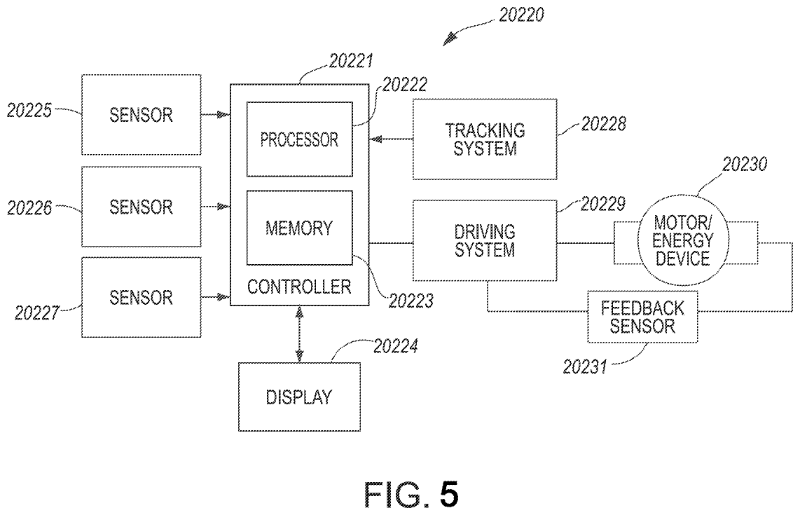

FIG. 5 illustrates a logical diagram of a control system 20220 of a surgical instrument or a surgical tool in accordance with one or more aspects of the present disclosure. The surgical is or the surgical tool may be configurable. The surgical instrument may include surgical fixtures specific to the procedure at-hand, such as imaging devices, surgical staplers, energy devices, endocutter devices, or the like. For example, the surgical instrument may include any of a powered stapler, a powered stapler generator, an energy device, an advanced energy device, an advanced energy jaw device, an endocutter clamp, an energy device generator, an in-operating-room imaging system, a smoke evacuator, a suction-irrigation device, an insufflation system, or the like. The system 20220 may comprise a control circuit. The control circuit may include a microcontroller 20221 comprising a processor 20222 and a memory 20223. One or more of sensors 20225, 20226, 20227, for example, provide real-time feedback to the processor 20222. A motor 20230, driven by a motor driver 20229, operably couples a longitudinally movable displacement member to drive the I-beam knife element. A tracking system 20228 may be configured to determine the position of the longitudinally movable displacement member. The position information may be provided to the processor 20222, which can be programmed or configured to determine the position of the longitudinally movable drive member as well as the position of a firing member, firing bar, and I-beam knife element. Additional motors may be provided at the tool driver interface to control I-beam firing, closure tube travel, shaft rotation, and articulation. A display 20224 may display a variety of operating conditions of the instruments and may include touch screen functionality for data input. Information displayed on the display 20224 may be overlaid with images acquired via endoscopic imaging modules.

The microcontroller 20221 may be any single-core multicore processor such as those known under the trade name ARM Cortex by Texas Instruments. In one aspect, the main microcontroller 20221 may be an LM4P230H5QR ARM Cortex-M4F Processor Core, available from Texas instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 20221 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The microcontroller 20221 may be programmed to perform various functions such as precise control over the speed and position of the knife and articulation systems. In one aspect, the microcontroller 20221 may include a processor 20222 and a memory 20223. The electric motor 20230 may be a brushed direct current (DC) motor with a gearbox and mechanical links to an articulation or knife system. In one aspect, a motor driver 20229 may be an A3941 available from Allegro Microsystems, Inc. Other motor drivers may be readily substituted for use in the tracking system 20228 comprising an absolute positioning system. A detailed description of an absolute positioning system is described in U.S. Patent Application Publication No. 2017/0296213, titled SYSTEMS AND METHODS FOR CONTROLLING A SURGICAL STAPLING AND CUTTING INSTRUMENT, which published on Oct. 19, 2017, which is herein incorporated by reference in its entirety.

The microcontroller 20221 may be programmed to provide precise control over the speed and position of displacement members and articulation systems. The microcontroller 20221 may be configured to compute a response in the software of the microcontroller 20221. The computed response may be compared to a measured response of the actual system to obtain an “observed” response, which is used for actual feedback decisions. The observed response may be a favorable, tuned value that balances the smooth, continuous nature of the simulated response with the measured response, which can detect outside influences on the system.

The motor 20230 may be controlled by the motor driver 20229 and can be employed by the firing system of the surgical instrument or tool. In various forms, the motor 20230 may be a brushed DC driving motor having a maximum rotational speed of approximately 25,000 RPM. In some examples, the motor 20230 may include a brushless motor, a cordless motor, a synchronous motor, a stepper motor, or any other suitable electric motor. The motor driver 20229 may comprise an H-bridge driver comprising field-effect transistors (FETs), for example. The motor 20230 can be powered by a power assembly releasably mounted to the handle assembly or tool housing for supplying control power to the surgical instrument or tool. The power assembly may comprise a battery which may include a number of battery cells connected in series that can be used as the power source to power the surgical instrument or tool. In certain circumstances, the battery cells of the power assembly may be replaceable and/or rechargeable. In at least one example, the battery cells can be lithium-ion batteries which can be couplable to and separable from the power assembly.

The motor driver 20229 may be an A3941 available from Allegro Microsystems, Inc. A3941 may be a full-bridge controller for use with external N-channel power metal-oxide semiconductor field-effect transistors (MOSFETs) specifically designed for inductive loads, such as brush DC motors. The driver 20229 may comprise a unique charge pump regulator that can provide full (>10 V) gate drive for battery voltages down to 7 V and can allow the A3941 to operate with a reduced gate drive, down to 5.5 V. A bootstrap capacitor may be employed to provide the above battery supply voltage required for N-channel MOSFETs. An internal charge pump for the high-side drive may allow DC (100% duty cycle) operation. The full bridge can be driven in fast or slow decay modes using diode or synchronous rectification. In the slow decay mode, current recirculation can be through the high-side or the low-side FETs. The power FETs may be protected from shoot-through by resistor-adjustable dead time. Integrated diagnostics provide indications of undervoltage, overtemperature, and power bridge faults and can be configured to protect the power MOSFETs under most short circuit conditions. Other motor drivers may be readily substituted for use in the tracking system 20228 comprising an absolute positioning system.

The tracking system 20228 may comprise a controlled motor drive circuit arrangement comprising a position sensor 20225 according to one aspect of this disclosure. The position sensor 20225 for an absolute positioning system may provide a unique position signal corresponding to the location of a displacement member. In some examples, the displacement member may represent a longitudinally movable drive member comprising a rack of drive teeth for meshing engagement with a corresponding drive gear of a gear reducer assembly. In some examples, the displacement member may represent the firing member, which could be adapted and configured to include a rack of drive teeth. In some examples, the displacement member may represent a firing bar or the I-beam, each of which can be adapted and configured to include a rack of drive teeth. Accordingly, as used herein, the term displacement member can be used generically to refer to any movable member of the surgical instrument or tool such as the drive member, the firing member, the firing bar, the I-beam, or any element that can be displaced. In one aspect, the longitudinally movable drive member can be coupled to the firing member, the firing bar, and the I-beam. Accordingly, the absolute positioning system can, in effect, track the linear displacement of the I-beam by tracking the linear displacement of the longitudinally movable drive member. In various aspects, the displacement member may be coupled to any position sensor 20225 suitable for measuring linear displacement. Thus, the longitudinally movable drive member, the firing member, the firing bar, or the I-beam, or combinations thereof, may be coupled to any suitable linear displacement sensor. Linear displacement sensors may include contact or non-contact displacement sensors. Linear displacement sensors may comprise linear variable differential transformers (LVDT), differential variable reluctance transducers (DVRT), a slide potentiometer, a magnetic sensing system comprising a movable magnet and a series of linearly arranged Hall effect sensors, a magnetic sensing system comprising a fixed magnet and a series of movable, linearly arranged Hall effect sensors, an optical sensing system comprising a movable light source and a series of linearly arranged photo diodes or photo detectors, an optical sensing system comprising a fixed light source and a series of movable linearly, arranged photodiodes or photodetectors, or any combination thereof.

The electric motor 20230 can include a rotatable shaft that operably interfaces with a gear assembly that is mounted in meshing engagement with a set, or rack, of drive teeth on the displacement member. A sensor element may be operably coupled to a gear assembly such that a single revolution of the position sensor 20225 element corresponds to some linear longitudinal translation of the displacement member. An arrangement of gearing and sensors can be connected to the linear actuator, via a rack and pinion arrangement, or a rotary actuator, via a spur gear or other connection. A power source may supply power to the absolute positioning system and an output indicator may display the output of the absolute positioning system. The displacement member may represent the longitudinally movable drive member comprising a rack of drive teeth formed thereon for meshing engagement with a corresponding drive gear of the gear reducer assembly. The displacement member may represent the longitudinally movable firing member, firing I-beam, or combinations thereof.

A single revolution of the sensor element associated with the position sensor 20225 may be equivalent to a longitudinal linear displacement d1 of the displacement member, where d1 is the longitudinal linear distance that the displacement member moves from point “a” to point “b” after a single revolution of the sensor element coupled to the displacement member. The sensor arrangement may be connected via a gear reduction that results in the position sensor 20225 completing one or more revolutions for the full stroke of the displacement member. The position sensor 20225 may complete multiple revolutions for the full stroke of the displacement member.

A series of switches, where n is an integer greater than one, may be employed alone or in combination with a gear reduction to provide a unique position signal for more than one revolution of the position sensor 20225. The state of the switches may be fed back to the microcontroller 20221 that applies logic to determine a unique position signal corresponding to the longitudinal linear displacement d1+d2+. . . dn of the displacement member. The output of the position sensor 20225 is provided to the microcontroller 20221. The position sensor 20225 of the sensor arrangement may comprise a magnetic sensor, an analog rotary sensor like a potentiometer, or an array of analog Hall-effect elements, which output a unique combination of position signals or values.

The position sensor 0225 may comprise any number of magnetic sensing elements, such as, for example, magnetic sensors classified according to whether they measure the total magnetic field or the vector components of the magnetic field. The techniques used to produce both types of magnetic sensors may encompass many aspects of physics and electronics. The technologies used for magnetic field sensing may include search coil, fluxgate, optically pumped, nuclear precession, SQUID, Hall-effect, anisotropic magnetoresistance, giant magnetoresistance, magnetic tunnel junctions, giant magnetoimpedance, magnetostrictive/piezoelectric composites, magnetodiode, magnetotransistor, fiber-optic, magneto-optic, and microelectromechanical systems-based magnetic sensors, among others.

The position sensor 20225 for the tracking system 20228 comprising an absolute positioning system may comprise a magnetic rotary absolute positioning system. The position sensor 20225 may be implemented as an AS5055EQFT single-chip magnetic rotary position sensor available from Austria Microsystems, AG. The position sensor 20225 is interfaced with the microcontroller 20221 to provide an absolute positioning system. The position sensor 20225 may be a low-voltage and low-power component and may include four Hall-effect elements in an area of the position sensor 20225 that may be located above a magnet. A high-resolution ADC and a smart power management controller may also be provided on the chip. A coordinate rotation digital computer (CORDIC) processor, also known as the digit-by-digit method and Volder's algorithm, may be provided to implement a simple and efficient algorithm to calculate hyperbolic and trigonometric functions that require only addition, subtraction, bit-shift, and table lookup operations. The angle position, alarm bits, and magnetic field information may be transmitted over a standard serial communication interface, such as a serial peripheral interface (SPI) interface, to the microcontroller 20221. The position sensor 20225 may provide 12 or 14 bits of resolution. The position sensor 20225 may be an AS5055 chip provided in a small QFN 16-pin 4×4×0.85 mm package. The tracking system 20228 comprising an absolute positioning system may comprise and/or be programmed to implement a feedback controller, such as a PID, state feedback, and adaptive controller. A power source converts the signal from the feedback controller into a physical input to the system: in this case the voltage. Other examples include a PWM of the voltage, current, and force. Other sensor(s) may be provided to measure physical parameters of the physical system in addition to the position measured by the position sensor 20225. In some aspects, the other sensor(s) can include sensor arrangements such as those described in U.S. Pat. No. 9,345,481, titled STAPLE CARTRIDGE TISSUE THICKNESS SENSOR SYSTEM, which issued on May 24, 2016, which is herein incorporated by reference in its entirety; U.S. Patent Application Publication No. 2014/0263552, titled STAPLE CARTRIDGE TISSUE THICKNESS SENSOR SYSTEM, which published on Sep. 18, 2014, which is herein incorporated by reference in its entirety; and U.S. patent application Ser. No. 15/628,175, titled TECHNIQUES FOR ADAPTIVE CONTROL OF MOTOR VELOCITY OF A SURGICAL STAPLING AND CUTTING INSTRUMENT, filed Jun. 20, 2017, which is herein incorporated by reference in its entirety. In a digital signal processing system, an absolute positioning system is coupled to a digital data acquisition system where the output of the absolute positioning system will have a finite resolution and sampling frequency. The absolute positioning system may comprise a compare-and-combine circuit to combine a computed response with a measured response using algorithms, such as a weighted average and a theoretical control loop, that drive the computed response towards the measured response. The computed response of the physical system may take into account properties like mass, inertia, viscous friction, inductance resistance, etc., to predict what the states and outputs of the physical system will be by knowing the input.

The absolute positioning system may provide an absolute position of the displacement member upon power-up of the instrument, without retracting or advancing the displacement member to a reset (zero or home) position as may be required with conventional rotary encoders that merely count the number of steps forwards or backwards that the motor 20230 has taken to infer the position of a device actuator, drive bar, knife, or the like.

A sensor 20226, such as, for example, a strain gauge or a micro-strain gauge, may be configured to measure one or more parameters of the end effector, such as, for example, the amplitude of the strain exerted on the anvil during a clamping operation, which can be indicative of the closure forces applied to the anvil. The measured strain may be converted to a digital signal and provided to the processor 20222. Alternatively, or in addition to the sensor 20226, a sensor 20227, such as, for example, a load sensor, can measure the closure force applied by the closure drive system to the anvil. The sensor 20227, such as, for example, a load sensor, can measure the firing force applied to an I-beam in a firing stroke of the surgical instrument or tool. The I-beam is configured to engage a wedge sled, which is configured to upwardly cam staple drivers to force out staples into deforming contact with an anvil. The I-beam also may include a sharpened cutting edge that can be used to sever tissue as the I-beam is advanced distally by the firing bar. Alternatively, a current sensor 20231 can be employed to measure the current drawn by the motor 20230. The force required to advance the firing member can correspond to the current drawn by the motor 20230, for example. The measured force may be converted to a digital signal and provided to the processor 20222.

For example, the strain gauge sensor 20226 can be used to measure the force applied to the tissue by the end effector. A strain gauge can be coupled to the end effector to measure the force on the tissue being treated by the end effector. A system for measuring forces applied to the tissue grasped by the end effector may comprise a strain gauge sensor 20226, such as, for example, a micro-strain gauge, that can be configured to measure one or more parameters of the end effector, for example. In one aspect, the strain gauge sensor 20226 can measure the amplitude or magnitude of the strain exerted on a jaw member of an end effector during, a clamping operation, which can be indicative of the tissue compression. The measured strain can be converted to a digital signal and provided to a processor 20222 of the microcontroller 20221. A load sensor 20227 can measure the force used to operate the knife element, for example, to cut the tissue captured between the anvil and the staple cartridge. A magnetic field sensor can be employed to measure the thickness of the captured tissue. The measurement of the magnetic field sensor also may be converted to a digital signal and provided to the processor 20222.

The measurements of the tissue compression, the tissue thickness, and/or the force required to close the end effector on the tissue, as respectively measured by the sensors 20226, 20227, can be used by the microcontroller 20221 to characterize the selected position of the firing member and/or the corresponding value of the speed of the firing member. In one instance, a memory 20223 may store a technique, an equation, and/or a lookup table which can be employed by the microcontroller 20221 in the assessment.

The control system 20220 of the surgical instrument or tool also may comprise wired or wireless communication circuits to communicate with the surgical hub 20065 as shown in FIG. 4.

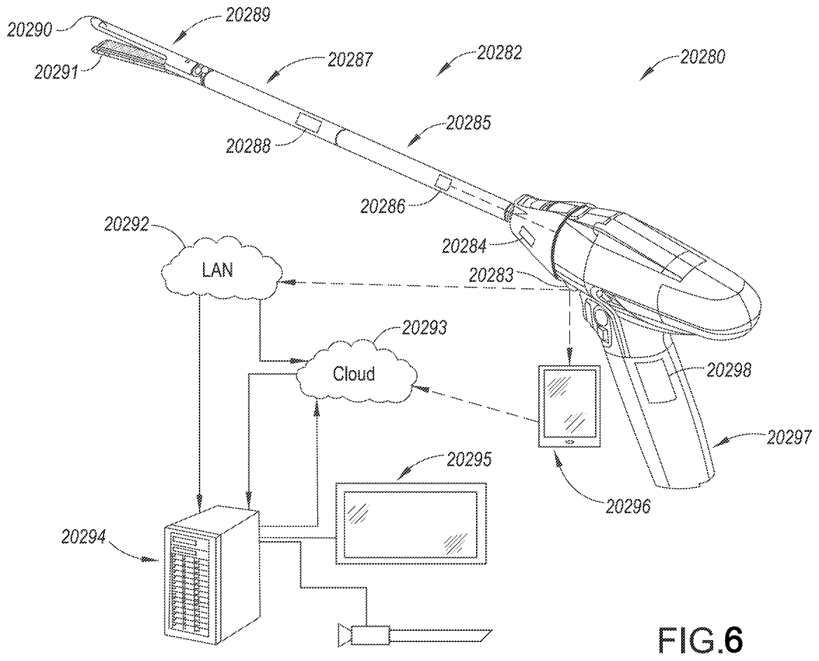

FIG. 6 illustrates an example surgical system 20280 in accordance with the present disclosure and may include a surgical instrument 20282 that can be in communication with a console 20294 or a portable device 20296 through a local area network 20292 and/or a cloud network 20293 via a wired and/or wireless connection. The console 20294 and the portable device 20296 may be any suitable computing device. The surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

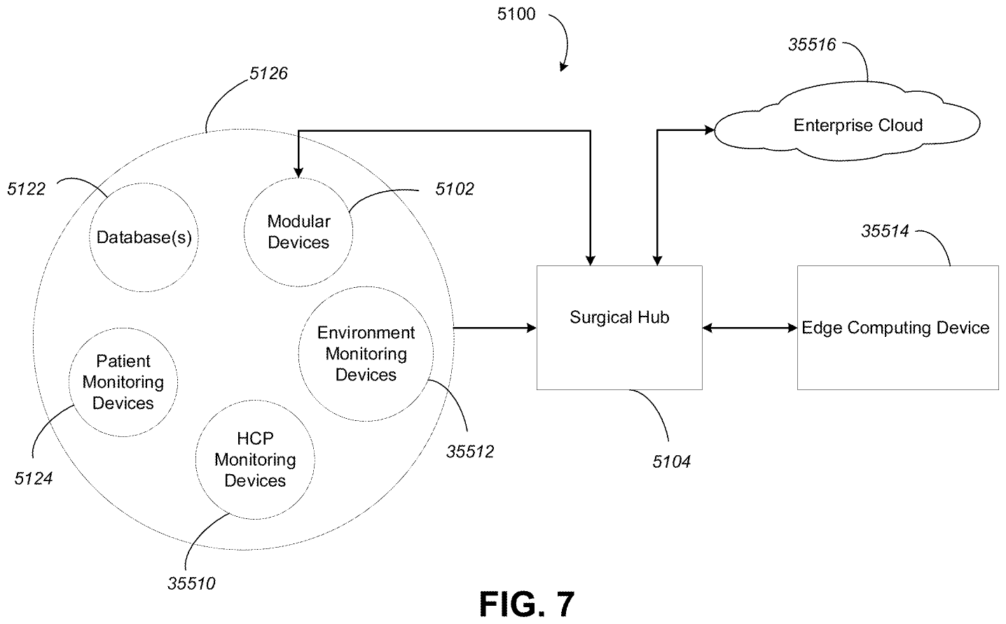

FIG. 7 illustrates a diagram of a situationally aware surgical system 5100, in accordance with at least one aspect of the present disclosure. The data sources 5126 may include, for example, the modular devices 5102 (which can include sensors configured to detect parameters associated with the patient, HCPs and environment and/or the modular device itself), databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The surgical hub 5104 can be configured to derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which is the hardware and/or programming associated with the surgical hub 5104 that derives contextual information pertaining to the surgical procedure from the received data and/or a surgical plan information received from the edge computing system 35514 or an enterprise cloud server 35516.

The situational awareness system of the surgical hub 5104 can be configured to derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. A machine learning system can be trained to accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a further machine learning system, lookup table, or other such system, which generates or retrieves one or more control adjustments for one or more modular devices 5102 when provided the contextual information as input.

A surgical hub 5104 incorporating a situational awareness system can provide a number of benefits for the surgical system 5100. One benefit may include improving the interpretation of sensed and collected data, which would in turn improve the processing accuracy and/or the usage of the data during the course of a surgical procedure. To return to a previous example, a situationally aware surgical hub 5104 could determine what type of tissue was being operated on; therefore, when an unexpectedly high force to close the surgical instrument's end effector is detected, the situationally aware surgical hub 5104 could correctly ramp up or ramp down the motor of the surgical instrument for the type of tissue.

The type of tissue being operated can affect the adjustments that are made to the compression rate and load thresholds of a surgical stapling and cutting instrument for a particular tissue gap measurement. A situationally aware surgical hub 5104 could infer whether a surgical procedure being performed is a thoracic or an abdominal procedure, allowing the surgical hub 5104 to determine whether the tissue clamped by an end effector of the surgical stapling and cutting is lung (for a thoracic procedure) or stomach (for an abdominal procedure) tissue. The surgical hub 5104 could then adjust the compression rate and load thresholds of the surgical stapling and cutting instrument appropriately for the type of tissue.

The type of body cavity being operated in during an insufflation procedure can affect the function of a smoke evacuator. A situationally aware surgical hub 5104 could determine whether the surgical site is under pressure (by determining that the surgical procedure is utilizing insufflation) and determine the procedure type. As a procedure type can be generally performed in a specific body cavity, the surgical hub 5104 could then control the motor rate of the smoke evacuator appropriately for the body cavity being operated in. Thus, a situationally aware surgical hub 5104 could provide a consistent amount of smoke evacuation for both thoracic and abdominal procedures.

The type of procedure being performed can affect the optimal energy level for an ultrasonic surgical instrument or radio frequency (RF) electrosurgical instrument to operate at. Arthroscopic procedures, for example, may require higher energy levels because the end effector of the ultrasonic surgical instrument or RF electrosurgical instrument is immersed in fluid. A situationally aware surgical hub 5104 could determine whether the surgical procedure is an arthroscopic procedure. The surgical hub 5104 could then adjust the RF power level or the ultrasonic amplitude of the generator (e.g., “energy level”) to compensate for the fluid filled environment. Relatedly, the type of tissue being operated on can affect the optimal energy level for an ultrasonic surgical instrument or RF electrosurgical instrument to operate at. A situationally aware surgical hub 5104 could determine what type of surgical procedure is being performed and then customize the energy level for the ultrasonic surgical instrument or RF electrosurgical instrument, respectively, according to the expected tissue profile for the surgical procedure. Furthermore, a situationally aware surgical hub 5104 can be configured to adjust the energy level for the ultrasonic surgical instrument or RF electrosurgical instrument throughout the course of a surgical procedure, rather than just on a procedure-by-procedure basis. A situationally aware surgical hub 5104 could determine what step of the surgical procedure is being performed or will subsequently be performed and then update the control algorithms for the generator and/or ultrasonic surgical instrument or RF electrosurgical instrument to set the energy level at a value appropriate for the expected tissue type according to the surgical procedure step.

In examples, data can be drawn from additional data sources 5126 to improve the conclusions that the surgical hub 5104 draws from one data source 5126. A situationally aware surgical hub 5104 could augment data that it receives from the modular devices 5102 with contextual information that it has built up regarding the surgical procedure from other data sources 5126. For example, a situationally aware surgical hub 5104 can be configured to determine whether hemostasis has occurred (e.g., whether bleeding at a surgical site has stopped) according to video or image data received from a medical imaging device. The surgical hub 5104 can be further configured to compare a physiologic measurement (e.g., blood pressure sensed by a BP monitor communicably connected to the surgical hub 5104) with the visual or image data of hemostasis (e.g., from a medical imaging device communicably coupled to the surgical hub 5104) to make a determination on the integrity of the staple line or tissue weld. The situational awareness system of the surgical hub 5104 can consider the physiological measurement data to provide additional context in analyzing the visualization data. The additional context can be useful when the visualization data may be inconclusive or incomplete on its own.

For example, a situationally aware surgical hub 5104 could proactively activate the generator to which an RF electrosurgical instrument is connected if it determines that a subsequent step of the procedure requires the use of the instrument. Proactively activating the energy source can allow the instrument to be ready for use as soon as the preceding step of the procedure is completed.

The situationally aware surgical hub 5104 could determine whether the current or subsequent step of the surgical procedure requires a different view or degree of magnification on the display according to the feature(s) at the surgical site that the surgeon is expected to need to view. The surgical hub 5104 could proactively change the displayed view (supplied by, e.g., a medical imaging device for the visualization system) accordingly so that the display automatically adjusts throughout the surgical procedure.

The situationally aware surgical hub 5104 could determine which step of the surgical procedure is being performed or will subsequently be performed and whether particular data or comparisons between data will be required for that step of the surgical procedure. The surgical hub 5104 can be configured to automatically call up data screens based upon the step of the surgical procedure being performed, without waiting for the surgeon to ask for the particular information.

Errors may be checked during the setup of the surgical procedure or during the course of the surgical procedure. For example, the situationally aware surgical hub 5104 could determine whether the operating theater is setup properly or optimally for the surgical procedure to be performed. The surgical hub 5104 can be configured to determine the type of surgical procedure being performed, retrieve the corresponding checklists, product location, or setup needs (e.g., from a memory), and then compare the current operating theater layout to the standard layout for the type of surgical procedure that the surgical hub 5104 determines is being performed. In some exemplifications, the surgical hub 5104 can compare the list of items for the procedure and/or a list of devices paired with the surgical hub 5104 to a recommended or anticipated manifest of items and/or devices for the given surgical procedure. If there are any discontinuities between the lists, the surgical hub 5104 can provide an alert indicating that a particular modular device 5102, patient monitoring device 5124, HCP monitoring devices 35510, environment monitoring devices 35512, and/or other surgical item is missing. In some examples, the surgical hub 5104 can determine the relative distance or position of the modular devices 5102 and patient monitoring, devices 5124 via proximity sensors, for example. The surgical hub 5104 can compare the relative positions of the devices to a recommended or anticipated layout for the particular surgical procedure. If there are any discontinuities between the layouts, the surgical hub 5104 can be configured to provide an alert indicating that the current layout for the surgical procedure deviates from the recommended layout.

The situationally aware surgical hub 5104 could determine whether the surgeon (or other HCP(s)) was making an error or otherwise deviating from the expected course of action during the course of a surgical procedure. For example, the surgical hub 5104 can be configured to determine the type of surgical procedure being performed, retrieve the corresponding list of steps or order of equipment usage (e.g., from a memory), and then compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

Surgical autonomous systems, devices, and methods may include aspects of integration with other medical equipment, data sources, processes, and institutions. Surgical autonomous systems, devices, and methods may include aspects of integration with a computer-implemented interactive surgical system and/or with one or more elements of a computer-implemented interactive surgical system, for example. Surgical system, surgical autonomous system, and autonomous surgical system may be interchangeably as described herein.

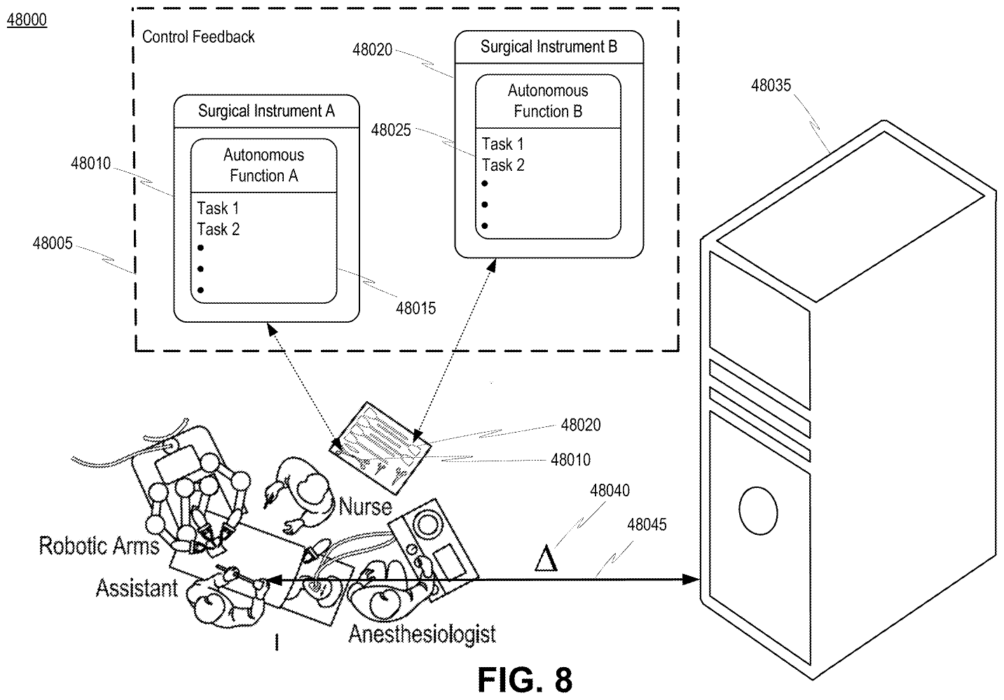

Referring to FIG. 8, an overview of the surgical autonomous system 48000 may be provided. Surgical instrument A 48010 and/or surgical instrument B 48020 may be used in a surgical procedure as part of the surgical system 48000. The surgical hub 48035 may be configured to coordinate information flow to a display of the surgical instrument. For example, the surgical hub may be described in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Example surgical instruments that are suitable for use with the surgical system 48000 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

FIG. 8 shows an example of a surgical autonomous system 48000. The system 48000 may be used to perform a surgical procedure on a patient who is lying down on an operating table in a surgical operating room. A robotic system may be used in the surgical procedure as a part of the surgical system. For example, the robotic system may be described in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. The robotic hub may be used to process the images of the surgical site for subsequent display to the surgeon through the surgeon's console.

Other types of robotic systems may be readily adapted for use with the surgical system 48000. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety,

In various aspects, an imaging device may be used in the surgical system and may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum of luminous spectrum, is that portion of the electromagnetic spectrum that is visible to (e.g., can be detected by) the human eye and may be referred to as visible light or simply light. A typical human eye will respond to wavelengths in air that are from about 380 nm to about 750 nm.

The invisible spectrum (e.g., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device may be configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that conies in contact with the patient or penetrates the sterile field, including the imaging device and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

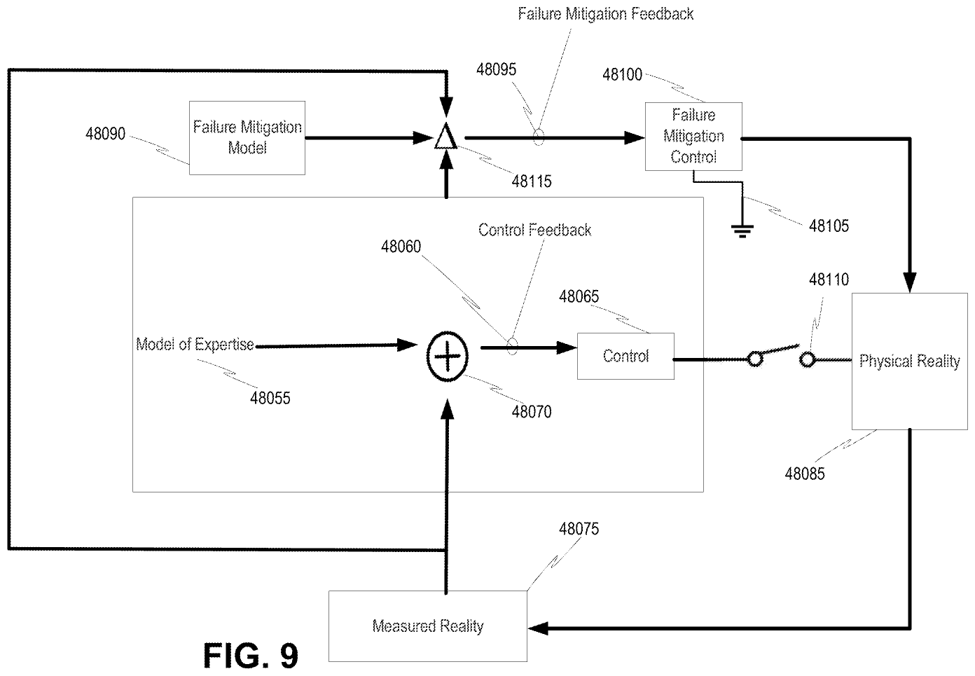

Surgical instrument A 48010 and/or surgical instrument B 48020 may comprise one or more capabilities (e.g., surgical instrument A comprises capabilities B, Z, and D and surgical instrument comprises capabilities C, F, and E). The capabilities may be associated with features (e.g., autonomous function A 48015 associated with surgical instrument A and autonomous function B 48025 associated with surgical instrument B) that the surgical instrument is capable of performing. Examples of the features may be described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example. For example, if the surgical instrument is an endocutter, one of the capabilities may be resecting tissue (e.g., tissue surrounding a colon when the surgeon in performing a colorectomy). The capabilities may comprise surgical tasks as described with respect to FIG. 9. For example, the capability of resecting tissue may comprise controlling an energy source, cutting, stapling, knob orientation, body orientation, body position, anvil jaw force, reload alignment slot management, and/or the like.

Surgical instrument A and/or B may be associated with control feedback or failure mitigation feedback, as described herein. For example, an autonomy level with the least manual input may be associated with the control feedback. For example, an autonomy level with increased manual input may be associated with the failure mitigation feedback.

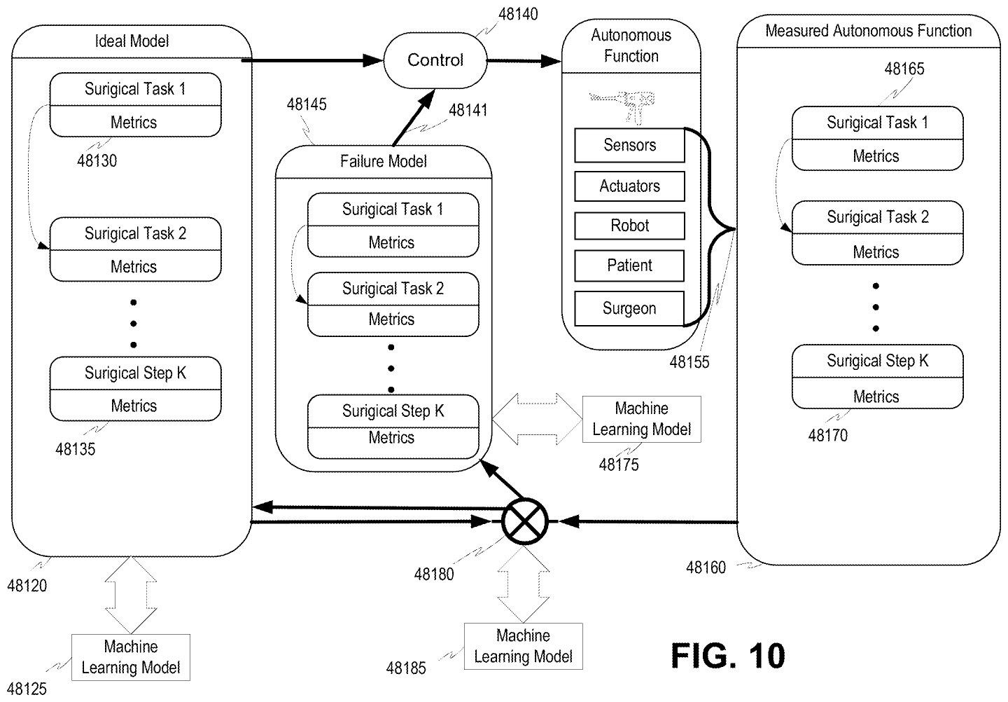

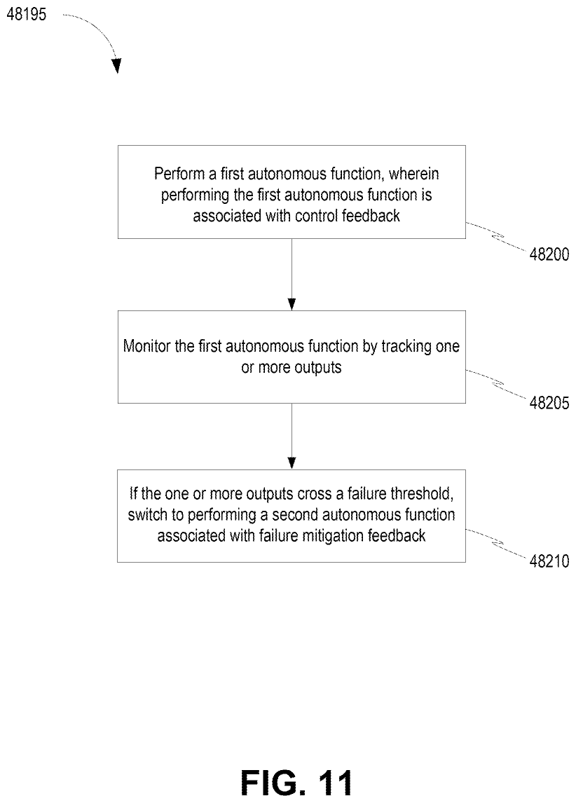

Data may be generated (e.g., by a monitoring module located at the surgical hub 48035 or locally by the surgical instrument as described with respect to FIG. 11) based on the performance of surgical instrument A and/or B. The data may be relevant to how the current level of autonomy at which the surgical instrument is operating is doing in terms of performance. For example, the data may be associated with physical measurement physiological measurements, and/or the like as described with respect to FIGS. 10 and 11. The measurements are described in greater detail under the heading “Monitoring Of Adjusting A Surgical Parameter Based On Biomarker Measurements” in U.S. patent application Ser. No. 17/156, 28, filed Nov. 10, 2021, the disclosure of which is herein incorporated by reference in its entirety.

An indication 48045 of the data may be transmitted to the surgical hub 48035, for example, where it may be evaluated. In examples, the indication 48045 may be transmitted to a cloud service (e.g., Amazon Web Services) as described herein. The data may be used as input in an analysis module, for example, to check if the performance of the surgical instrument falls within a satisfactory range (e.g., crosses a failure threshold 48040). The indication may be associated with switching from using the control feedback 48005 to the failure mitigation feedback.

FIG. 9 shows the relationship between control feedback 48060 and failure mitigation feedback 48095. As shown in FIG. 9, the device may include a model of expertise 48055. The model of expertise 48055 may provide parameter(s) such that a surgical instrument may be operated autonomously. For example, the surgical instrument may perform (e.g., be capable of performing) one or more surgical tasks autonomously. For example, the model of expertise 48055 may be linked to a machine learning algorithm. The machine learning algorithm, as described herein, may include a neural network structure that may generate parameter(s) (e.g., weights of parameters) to be used, for example, on a script located internally in the surgical instrument. In examples, the script may be executed remotely, for example, at the surgical hub. The parameters outputted by the model of expertise 48055 and executed by the script may be dynamic. The script may be used to execute one or more actuators, as described with respect to FIG. 6, located on the surgical instrument. For example, the one or more actuators may be linear actuators and/or rotary actuators.

The actuators may be servomotors located on the surgical instrument. The script may be generated using a machine learning algorithm with adjustable parameters and may be used in connection with hardware, such as linear and rotary actuators, to allow the surgical instrument to perform a surgical task. For example, the script may produce control feedback 48060, which may be used as input to the actuators. The actuators may allow the surgical instrument to be controlled autonomously via the script. For example, the actuators may receive the adjustable parameters as input and use the parameters to perform the surgical task autonomously.

Physical reality measurement(s) 48085 may be obtained. For example, physical reality measurements 48085 may relate to one or more sensors placed on the surgical instrument performing the surgical task. In examples, physical reality measurements 48085 may be obtained and/or may be generated from a patient wearing wearable sensors that measure the patient's biomarkers. In examples, the physical reality measurements 48085 may be generated from a surgeon wearing wearable sensors that measure the surgeon's biomarkers and other health markers.

The physical reality measurements 48085 may be sent to a surgical hub and may be analyzed at the surgical hub. In examples, the physical reality measurements 48085 may be generated locally at the surgical instrument, for example, by the surgical instrument processor, as described herein. In examples, the surgical instrument and/or surgical hub may include an analysis module that may analyze the physical reality measurements 48085. If the physical reality measurements 48085 are generated by a remote source, such as a surgical hub, the physical reality measurements 48085 may be sent via a message to the surgical instrument. In examples, the message may pass through a surgical application program interface (API). The physical reality measurements 48085, or in other words, the data related to the physical reality measurements may be compared to model measurements (e.g., model data) as described herein.

The physical reality measurements 48085 may be translated into measured reality data 48075. For example, the physical reality measurements 48085 may be passed through an analysis module located at the surgical hub or locally at the surgical instrument. The output of the analysis module may be data related to the measured reality. For example, the physical reality measurements 48085 may be raw data related to the position of the linear and/or rotary actuators of the surgical instrument. For example, this data may be in the form of voltage readings. In examples, an analog-to-digital converter (ADC) may be included and may transform the voltage readings into a bitstream, which may be used as the physical reality measurements 48085. This raw data may be sent to an analysis module located at the surgical hub or locally at the surgical instrument and the analysis module may output measured reality data 48075. Measured reality data 48075 may in a form better suited for comparison when compared to the raw physical reality measurements 48085.

The measured reality data 48075 may be compared to data associated with the model of expertise. For example, the model of expertise may include a data structure that holds model data. The model data may be referred to as with model reality data herein. This model reality data may be compared to measured reality data 48075 and a difference 48070 between the two may be generated. This difference 48070 between the model reality data and the measured reality data 48075 may be used as control feedback 48065 to adjust parameters that control the servomotors, such as the linear and rotary actuators described herein.

For example, the difference 48070 between the model reality data and the measured reality data may indicate that the speed at which the rotary actuators rotate is too high. The measure reality data 48075 associated with rotational speed may be greater than the model reality data associated with rotational speed for rotary actuator A positioned at a certain spot on the surgical instrument. Based on this indication, control feedback 48065 may be generated in which a parameter associated with the speed of the rotary actuator A may be decreased such that the speed of the rotary actuator is decreased.

The model of expertise 48055 may be used to generate the control feedback 48065 for the control of the actuators of the surgical instrument operating autonomously, e.g., as long as the difference 48070 between the modeled reality measurements data and the measured reality data 48075 falls within a threshold. If the difference 48070 between the two does not fall within a threshold 48115 (e.g., exceeds a threshold 48115), a failure mitigation model 48090 may be used (e.g., control loop grounded to zero 48110 and failure mitigation loop release from grounded zero 48105).

The failure mitigation model 48090 may generate parameters to be used by the surgical instrument operating autonomously. The parameters may be set to values associated with no risk. In examples, the failure mitigation model 48090 may turn the surgical instrument from autonomous to a manual setting. The surgical instrument using the failure mitigation parameters may control the linear and/or rotary actuators and other motor controls based on the failure mitigation feedback 48095.

Physical reality measurements 48085 may be generated to assess the performance of the failure mitigation control 48100. Similar to the model of expertise 48055, physical reality measurements 48085 may be in the form of raw data and may pass through an analysis module. The analysis module may generate measured reality data 48075, which may be used and compare against failure model data. The measured reality data 48075 that are associated with failure mitigation parameters may be compared to failure model reality data, for example, to assess the performance of the failure mitigation model 48090.

A notification of the switch from the model of expertise 48055 to the failure mitigation model 48090 may be sent via a message to a user of the surgical instrument. The message may include the parameters that are being set for the surgical instrument. In examples, when comparing the measured reality data 48075 to the model reality data, a heart rate of the patient may be compared. This data related to the heart rate may be checked against a threshold 48115. If the heart rate exceeds the threshold, the surgical instrument may switch from using a model of expertise script to a failure mitigation model script. The failure mitigation model 48090 may produce failure mitigation parameters. The parameters may be known to result in the surgical instrument being able to perform a surgical task with no risk involved.

FIG. 10 shows an example of performing an autonomous function with an ideal model 48120 and a failure model 48145 As shown in FIG. 10, an ideal model 48120 may include one or more surgical tasks. Ideal model 48120 and model of expertise may be used interchangeably herein. The ideal model 48120 may be generated by a surgical hub. For example, the ideal model 48120 may be generated by the surgical hub as described with respect to FIG. 9. The surgical tasks (e.g., each of the surgical tasks) of the ideal model, for example, surgical task one 48130, surgical task two, and surgical task K 48135, may be associated with one or more ideal metrics. The ideal metrics may be associated with ideal physiological and/or physical metrics related to the performance of the surgical task. For example, the surgical task may be performed autonomously. The ideal metrics may be related to physiological and/or physical expected conditions that the surgeon may expect to be present based on the surgical task. For example, the surgical task may be removing surrounding tissue from an organ, such as a colon. The ideal physical conditions and/or metrics for this surgical task may include the patient's heart rate being above a certain value and below a certain value (e.g., within an acceptable range). This may be considered the ideal heart rate range for mobilizing the colon. In examples, the surgical task may be resecting a tissue, which may involve the use of an endocutter. Resecting tissue may include one or more instrument capabilities being performed such as positioning the instrument body, controlling anvil jaw force, and managing reload alignment slot. These capabilities may be performed autonomously. For example, a first autonomous function may be associated with (e.g., may be a software module located within) the model of expertise as described herein. To perform the capabilities for the surgical task of tissue resection, the model of expertise may run the first autonomous function (e.g, the script of the autonomous function). Based on a failure mitigation threshold having been crossed, a second autonomous function may be called. The second autonomous function may be associated with (e.g., may be a software module located within) the failure mitigation model. To perform the capabilities for the surgical task of tissue resection, the model of expertise may switch from running the first autonomous function (e.g, the script of the first autonomous function) to running the second autonomous function (e.g, the script of the second autonomous function).

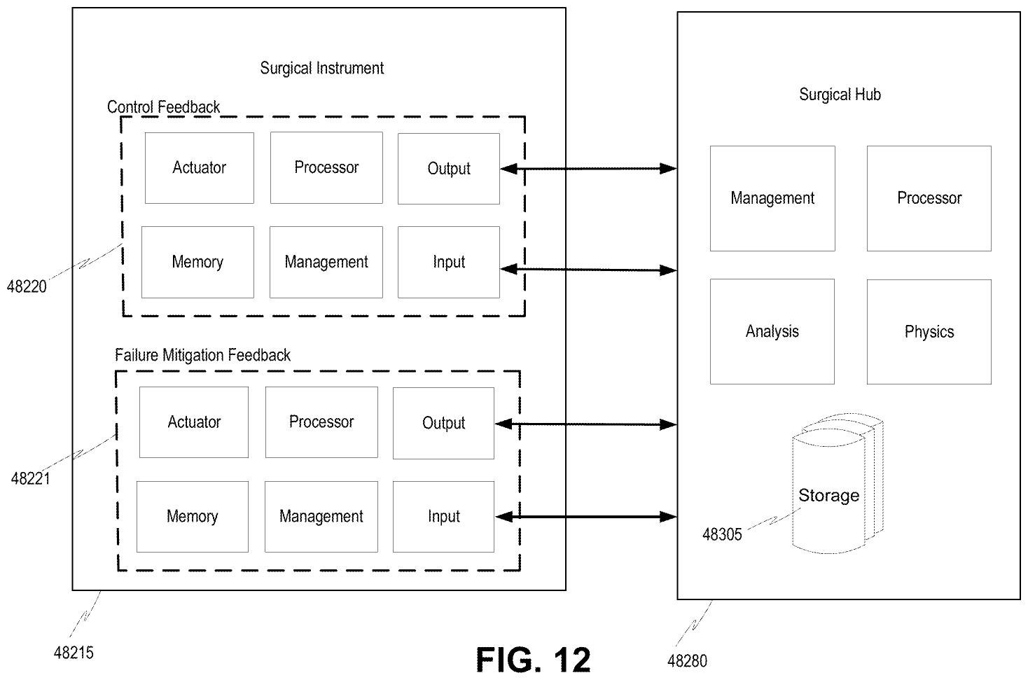

The physical and/or physiological ideal conditions may change based on the surgical task. For example, surgical task two may have different ideal physiological and/or physical conditions when compared to surgical task one. The physical metrics may include data generated from sensors from one or more actuators as described with respect to FIG. 9 and FIG. 12. In order for the surgical instrument to perform an autonomous function, the surgical instrument may perform surgical task one 48130, surgical task two, and/or surgical task K 48135. The ideal physical and/or physiological conditions may be compared to measured physiological and/or physical conditions, which, e.g., as shown with respect to the measured autonomous function 48160, the measured physiological and/or physical conditions may be generated 48155 from one or more of the following: sensors, actuators, data generated from robotic functions, data generated from the patient, such as physiological data, data generated from the surgeon, such as physiological surgeon data.

The data generated 48155 from the patient and/or surgeon may involve the use of wearable devices to measure biomarkers associated with the patient and/or surgeon. The wearables are described in greater detail under the heading “Monitoring Of Adjusting A Surgical Parameter Based On Biomarker Measurements” in U.S. patent application Ser. No. 17/156, 28, filed Nov. 10, 2021, the disclosure of which is herein incorporated by reference in its entirety.

This data may be generated 48155 and used as an input to gather the physiological and/or physical metrics. The data associated with the ideal physical and/or physiological metrics may be used and compared to the data generated from the measured physical and/or physiological conditions (e.g., for each of the surgical tasks such as surgical task 1 48165, surgical task 2, and surgical task K 48170 associated with the measured autonomous function 48160 and a difference between the two may be generated and compared to a threshold 48180, which may take place at the surgical hub, or locally at the surgical instrument, as describe with respect to FIG. 9 and FIG. 12.

Determining the threshold 48180, e.g., at which the difference between the ideal physical and/or physiological conditions and the measured physical and/or physiological conditions is compared to, may be generated using a machine learning model 48185, which may include a neural network structure. The neural network structure may use one or more of the ideal physical and/or physiological conditions as parameters when determining the appropriate threshold level 48180. If the threshold level 48180, e.g., which may be referred to as the failure mitigation threshold level 48180, as described with respect to FIG. 12, has been exceeded based on the difference between the ideal physiological and physical conditions and the measured physical and physiological conditions, the surgical instrument may switch to using a failure model 48145.

The failure model 48145 may involve the surgical instrument being associated with failure mitigation feedback. The failure model 48145 may include physiological and/or physical failure metrics such as failure physical and/or physiological conditions. Similar to the ideal model, these conditions may be compared to measured physiological and physical conditions in the described herein. Determining both the ideal physical and physiological conditions and the failure physical and physiological conditions may involve the use of a machine learning model 48175 as described herein. For example, the machine learning model 48175 associated with the failure mitigation model 47145 may determine failure mitigation metrics that eliminate or minimize any risk associated with performing one or more surgical tasks (e.g., surgical task 1, 2, and/or K).

The machine learning model 48125 associated with the ideal model may determine one or more physical and/or physiological metrics associated with an optimized performance of the surgical task. The optimized performance may consider costs and/or benefits associated with the surgical task. For example, the surgical task one may involve the surgeon mobilizing a patient's colon. During the surgical task, a surgeon's heart rate may have an ideal physiological measurement. A patient's heart rate may have an ideal physiological measurement. The position of the instrument may have an ideal physical measurement, which may be determined by the position of one or more actuators. In examples, this data may be analyzed and a value may be generated. In examples, each of the ideal data collected from physiological and/or physical conditions may be compared to respective measured physiological and/or physical conditions. For example, the surgeon's ideal heart rate may be compared to the surgeon's measured heart rate as determined during surgical task one of the autonomous function. The patient's heart rate may be compared to the measured patient's heart rate during surgical task one 48130. In examples, if one of the differences between the ideal model physical condition and the measured model physical condition and/or physiological condition exceeds the failure mitigation threshold 48180, the surgical instrument may switch from operating on control feedback 48140 to operating on failure mitigation feedback 48141. The failure mitigation feedback 48141 may be described with respect to FIG. 9. In examples, when switching from surgical task one 48130 to surgical task two, a set of ideal physical and/or physiological metrics may be generated by the ideal model, which may be located on the surgical instrument and/or on a surgical hub.

In examples, the surgical instrument may pull data from a remote source to generate ideal metrics associated with surgical task two, which may be based on historical data associated with surgical task two. The historical data may be based on a facility's historical data or may be based on a general historical data associated with surgical task two. The physical and/or physiological conditions and/or metrics associated with the physical and/or physiological conditions may be manually inputted by a surgeon or another user of the surgical instrument.

FIG. 11 shows an example of performing an autonomous function with control feedback and failure mitigation feedback. An overview 48195 of performing an autonomous function with control feedback and failure mitigation feedback may be provided. Autonomous function and autonomous surgical task may be used interchangeably herein.

At 48200, a first autonomous function may be performed. The first autonomous function may be associated with the model of expertise as described with respect to FIG. 9. For example, the model of the expertise may output the parameter(s) to be used as the autonomous function is performed. The autonomous function may perform a surgical task associated with a surgical instrument. The controls of the surgical instrument such as the actuators described with respect to FIG. 9 and FIG. 12 may be controlled by control feedback. The control feedback may update the parameters based on the current performance of the autonomous function.

The control feedback may be generated when comparing modeled reality data associated with the model of expertise with measured reality data that is generated based on the physical and/or physiological measurements associated with performing the surgical task. For example, the physical measurements may include the position of the actuator(s) located on the surgical instrument performing the surgical task.

At 48205, the first autonomous function, which may be associated with the model of expertise, may be monitored. For example, one or more outputs may be generated based on the performance of the autonomous function.

The outputs may be the physical measurements and may be in the form of raw data. The outputs may be fed into an analysis module either at a surgical hub or locally at the surgical instrument. The outputs may include data generated based on situational awareness, for example, surgical context data may be sent to the surgical hub. Data generated based on situation awareness may include any data suitable for characterizing the current presentation of the patient in view of the ongoing surgical procedure. For example, such data may include procedural stage, current operating room (OR) arrangement, patient vitals, instruments, engaged, and/or the like. The surgical hub may use the surgical context data when generating measured reality data. The surgical context data, for example, may be sent to analysis module along with physical measurement data.

The model of expertise may generate parameters by using a machine learning model. The parameters may be used to perform the autonomous function. When generating the parameters to be used for the autonomous function, the machine learning model may consider one or more of the following: historical data; surgical context; the type of the first autonomous function, the type of the second autonomous function, historical data associated with the first autonomous function, number of outputs, or the degree of the one or more parameters that have crossed the failure threshold. Threshold and failure threshold may be used interchangeably herein. The one or more of the following described herein may apply when determining whether the difference between the measured reality data and the model reality data has crossed the failure threshold. The first autonomous function may be monitored (e.g., as described with respect to FIG. 9, the analysis module may output measured reality data which may be compared to model reality data and a difference between the two may be produced). The difference between the two may be compared to a failure threshold.

At 48210, if it is determined that the failure threshold has been exceeded, the surgical instrument may switch to performing a second autonomous function. The second autonomous function may be associated with the failure mitigation model. The failure mitigation model may produce failure mitigation feedback which may result in adjusting the parameters to a value which results in the surgical instrument being able to perform the surgical task with no risk involved. In examples, a difference between the measured reality data and the model reality data may be generated for each physical measurement obtained from the model of expertise. A physical measurement related to the speed of a rotary actuator may be generated. This data may be processed in an analysis module. Measured reality data related to the speed of the rotary actuator may be outputted from the analysis module.

Model data related to the speed of the rotary actuator may be contained in the model of expertise as a data structure such as in a list. The speed of the rotary actuator from the model data structure may be compared to the actual speed of the rotary actuator in the measured reality data and a respective output may be produced based on the difference between the two and compared to a respective failure threshold. This may be performed for multiple physical measurements which may produce respective measured reality data, which may be compared to respective model reality data. The differences may be compared to respective thresholds (e.g., failure thresholds).

In examples, if one of the respective differences between the measured reality data and the model reality data crosses the respective threshold, the surgical instrument may switch from the first autonomous function to the second autonomous function. The number of differences that have to cross the output before switching to the second autonomous function may be manually inputted by a user of the surgical instrument. For example, a surgeon may set three as a number of times the failure threshold must be exceeded by respected differences before switching to the second autonomous function.

The second autonomous function may be associated with the failure mitigation function as described herein. The difference between the measured reality data and the model reality data may be a single value. For example, the model of expertise may include an analysis module that may assess the difference between each measured reality data and each model reality data and may come up with a value based on the difference between the two. In such a case, the single difference from all of the respective measured reality data and the model reality data may be compared to a single failure threshold. When the single failure threshold is exceeded, the surgical instrument may switch from the first autonomous function to a second autonomous function to perform the surgical task.

A failure magnitude may be generated based on the difference. For example, a failure magnitude may be based on the degree at which the difference between the measured reality data and the model reality data crossed the failure threshold. For example, if the difference between the measured reality data and the model reality data exceeds the failure threshold by a greater amount, the failure magnitude may be higher than if the difference exceeds the failure threshold at a lesser amount. The failure magnitude may be used when generating the failure mitigation parameters. For example, the failure mitigation parameters may be dynamic and may operate within a range. A higher failure magnitude may result in the surgical instrument using parameters that result in less risk in terms of the of the completion of the surgical task. In examples, the failure magnitude may be used to switch the surgical instrument from autonomous to manual.

In examples, failure magnitude may be used to terminate all operations related to the surgical task. The failure magnitude may be generated based on one or more of the following: surgical context; the type of the first autonomous function; the type of the second autonomous function; historical data associated with the first autonomous function; the number of outputs that cross the failure threshold; or the degree at which one or more of the outputs crossed the failure threshold. Outputs may mean the difference between the measured reality data and the model reality data. In examples, instead of generating a difference between the measured reality data and the model reality data, the measured reality data may be compared to the threshold. From this comparison, it may be determined whether the threshold was crossed and, in such a case, the surgical instrument may switch from the first autonomous function to the second autonomous function.

Weights may be assigned to the outputs (e.g., each of the outputs) of the analysis module and/or to the measured reality data (e.g., each of the measured reality data). The speed of the actuators may be assigned a higher weight when compared to the position of the sensors (e.g., potentiometers). Assigning the weight may be performed by the analysis module either locally at the surgical instrument or on the surgical hub. Assigning the weight may take place on a remote service such as a cloud service. For example, the cloud service may be the Amazon Web Services lambda function. When comparing whether the difference between the model reality data and the measured reality data crossed the failure threshold, the weights may be considered. For example, if the difference between the measured reality data associated with the speed of the actuators crossed the failure threshold, it may be more likely that the surgical instrument switches to the second autonomous function. If the difference between the model reality data and the measured reality data associated with the position of the actuators crossed the failure threshold, the surgical instrument may be less likely to switch to the second autonomous function. Assigning the weights may be based on one or more of the following: historical data, surgical context, type of the first autonomous function, type of the second autonomous function, historical data associated with the first autonomous function, the number of the outputs that cross failure threshold (e.g., number of differences between the measure reality data and the model reality data that crossed the threshold); or a degree of difference of the outputs crossing the failure threshold.

As described with respect to FIG, 9, a message may be sent to a user of the surgical instrument if the surgical instrument switches from the first autonomous function which is associated with the model of expertise and the second autonomous function, which is associated with the failure mitigation model. A set of recommendations may be included in the message to the user (e.g., surgeon). For example, the recommendations may include which measured reality data has exceeded the threshold and may suggest how the autonomous function may use failure mitigation feedback to prevent any complications. In examples, the surgical instrument may switch back to the first autonomous function which is the autonomous function associated with the model of expertise. These switches may occur multiple times during the performance of a surgical task. These switches may be based on the difference between the measured reality data and model reality data falling within or below a threshold. In examples, a risk assessment model may be included and used when determining whether to switch between autonomous functions.

Generating the recommendations may be based on the type of the first autonomous function, the type of the second autonomous function, historical data associated with the first autonomous function, the difference, the number outputs that cross the failure threshold, and/or the degree at which the outputs crossed the failure threshold. The model of expertise may initialize the parameters based on training data it received prior to performing the surgical task. The training data may be based on one or more of the following: surgical context, type of the first autonomous function, type of the second autonomous function, historical data associated with the first autonomous function, a number of outputs that crossed the failure threshold or a degree of the one or more outputs crossing the threshold.

FIG. 12 shows an example of the overview of the surgical instrument 48215 with control feedback 48220 and the failure mitigation feedback 48221 and the surgical hub 48280. As shown with respect to FIG. 12, a surgical instrument 48215 may be in communication with a surgical hub 48280. In examples, the surgical hub 48280 may be a third-party service such as an edge service on a cloud platform.

The surgical instrument 48215 may include control feedback 48220 and failure mitigation feedback 48221. The control feedback 48220 may be used while performing an autonomous function associated with a surgical task. For example, if the surgical task is mobilizing a colon, the autonomous function may be freeing the colon from surrounding tissue, which may involve the use of an endocutter. The control feedback 48220 may set, e.g., via a message to the surgical instrument 48215, the parameters for the endocutter to perform the autonomous function, e.g. freeing the colon from surrounding tissues. The control feedback 48220 may adjust the one or more parameters (e.g., the autonomous parameters).

The parameters may be used as input for the actuators, which may include the one or more actuators described with respect to FIG. 9. The parameters may be adjusted based on feedback from a machine-learning model. For example, an error rate may be measured based on the performance of the autonomous function and if the error rate exceeds a threshold as assessed by a machine-learning model, feedback may be sent to the surgical instrument to adjust the one or more autonomous parameters.

The control feedback 48220 may include a memory. The memory may store the one or more current parameters. The memory may store the parameters via a database as described with respect to FIG. 9 and FIG. 10. Control feedback 48220 may include a management module. The management module may be responsible for sending an update message to the memory if the one or more autonomous parameters are updated. The control feedback may include a processor which may be responsible for running the instructions to perform the autonomous function.

The processor may be in communication with the memory. The memory may include an instruction memory which the processor may read or write to. The control feedback 48220 may be associated with the normal operation of the autonomous function to perform the associated surgical task (e.g., autonomously). The control feedback may have one or more autonomy levels. Switching autonomy levels may result in adjusting the one or more autonomous parameters. The autonomy level may be stored in the memory along with the one or more autonomous parameters associated with the autonomy level.

Control feedback 48220 within the surgical instrument may generate output. The output may be related to the performance of the autonomous function. The output may include the one or more parameters of the autonomous function. The output may be generated and may be sent via a message to the surgical hub 48280. The surgical hub 48280 may perform analysis on the output. The surgical hub 48280 may send a response message. In such a case, the surgical instrument 48215 may receive input data from the surgical hub 48280 via the response message. The input data may include an indication to update the one or more autonomous parameters associated with the autonomous function.

The machine-learning model, as described herein, may be located within the surgical hub 48280. For example, the machine-learning model may be a module within the surgical hub 48280. Storage 48305 may be included in the surgical hub. In examples, the storage 48305 may be off-disk storage. A management module of the surgical hub 48280 may be responsible for sending data associated with the output data of the control feedback 48220 to the storage 48305. The output data, as described herein, may be associated with a performance of the autonomous function and/or the parameters of the autonomous function. Such data may be stored in the storage 48305 along with a timestamp of when the output data was generated.

The management module of the surgical hub 48280 may send such data to the storage 48305. In examples, the analysis module may be included in the surgical hub 48280. The analysis module may compare the performance of the autonomous function as generated by the output module to a threshold (e.g., a failure mitigation threshold). The analysis module may determine whether the output data exceeds the failure mitigation threshold. In such a case, if such data does exceed the failure mitigation threshold, the surgical hub 48280 may send a message to the surgical instrument 48215 to switch from using control feedback 48220 to failure mitigation feedback 48221. The failure mitigation feedback 48221 may be the failure mitigation feedback as described with respect to FIGS. 9, 10 and 11.

The failure mitigation feedback 48221 may include one or more actuators. The one or more actuators may be the same actuators as described with respect to the control feedback 48220. The parameters used for the one or more actuators may be failure mitigation parameters. The failure mitigation parameters may be set to a value where no risk or minimum risk is expected based on the performance of the autonomous function. For example, the failure mitigation parameters may include values that result in 100% success rate of the autonomous function. The failure mitigation parameters may be stored in a memory associated with the failure mitigation feedback 48221. A processor of the surgical instrument may request those values when performing the autonomous function. Failure mitigation feedback may be data generated by the failure mitigation model (e.g., using the script associated with the second autonomous function) that is sent (e.g., signaled) to the hardware of the surgical instrument, for example, to control the surgical instrument autonomously as it performs the surgical task. The data generated may be set to values that ensure no risk, or at least reduced risk, is involved in the autonomous performance of the surgical task.

The failure mitigation parameters may be used as input for the actuators to perform the surgical task at hand. The failure mitigation parameters may be sent from the surgical hub 48280. The performance of the surgical instrument 48215 that is using the failure mitigation parameters may generate output data associated with the performance, which may be sent to surgical hub 48280 and analyzed. The analysis module may assure no risk is involved in the performance of the autonomous function.

The surgical hub 48280 may detect that risk is still present based on the failure mitigation parameters and/or the performance of the failure mitigation feedback output. The surgical hub 48280 may adjust the failure mitigation parameters, which may be sent via a message and received as input by the surgical instrument 48215. The failure mitigation parameters may be updated in memory. The management module associated with the failure mitigation feedback 48221 may be responsible for updating the memory. In order to perform the autonomous function with failure mitigation feedback 48221, the processor may write or read to the memory associated with the failure mitigation feedback 48221.

Pro-active monitoring and/or reactions may be based on encountered issues (e.g., automated issue resolution). Autonomous function oversight monitoring and/or failure mitigation may be provided, which may include monitoring of an autonomous function, magnitude and/or level for identification of failures of an autonomous operation (e.g., portion of an autonomous function). An identified failure may have a determination of importance of the failure based on risk, redundancy, timing of occurrence, magnitude of occurrence, or impact of the occurrence. The identified failure may be followed up with an indication of the failure to the user and/or to a system outside of the hub system. The indication may result in active correction of the issue. The indication may be to the user, the facility, maintenance, the manufacturer, and/or complaint database. The indication may result in a mitigation of the failure, and the result of the mitigation may be part of the indication to one or more systems and/or users. The mitigation may reduce system performance, for example, to enable the conclusion of the step or procedure. It may be shut-down the affected portion of the system. It may include a substitution of an alternative means of monitoring and/or controlling the medical hardware. It may allow for a pause and/or hold of instrument motions, for example, until the system failure is resolved.

Reporting may be provided, which may include complaint and/or issue automatic documentation. Complaint and/or issue automatic documentation may include one or more of the following: a user guided form and/or template identification and assistant may be used in completion; complaint categorization and/or routing (e.g., manufacturer directed issues and/or facility directed issues); machine learning aggregation and sorting for trends; or automated reporting and/or summarization. Complaint and/or issue automatic documentation may include the surgical hub being fully aware if a device within the operating room theater has had malfunction or error condition. This information may be autonomously pulled and pushed into the complaint database for review. The information may be categorized a number of ways. For example, a way may be to have the hub look into the device records and determine the severity (e.g., DFMEA severity) of the failure and use this as a category. A way may be to use the hospital's risk systems to base the categorization against.

Automated recording and/or reporting of failures, their potential causes, associated operation circumstances, resolutions or mitigation, and user interactions or experiences may be due to the failure. The system may record (e.g., automatically record) the log data (e.g., all log data). Data may be stored to a database associated with that system. In examples, the system may record (e.g., automatically record) the log data for a given period of time. For example, for rollover recording log, the system may log (e.g., automatically log) all data (e.g., regardless of other severity, criticality, etc.) for a given period of time. As the time period elapses, the system may automatically start to overwrite the oldest data first to preserve memory space. For period of accountability, the system may automatically log all data for a given period of time, for example, that it may be legally required and may automatically discard the data after that period of time has elapsed. The data that has been identified may be exempted from the automatic discard feature. For patient outcome, the log data may be held from the time of surgery, for example, until the outcome of the patient has been ascertained. If an adverse event for that patient is identified, the data may be held and not removed. If no adverse events are identified, after a certain period of time post surgery, the log data may be discarded.

Backup and/or auditing of the automation post operation may be used to review and/or document automated functions and their impact on the user, procedure step, and/or device outcome. User monitoring of reactions and response to an autonomous operation may be provided. Overriding of the primary response may be provided. For example, steps that the surgeon deviates from may be flawed for review. For example, steps within the procedure that the system believes are a risk and the surgeon deems it acceptable may be annotated as an override by the user and the number of these triggers may be used as a trigger for auditing (e.g., AI review, peer review, variations away from the IFU, etc.). A robotic system may perform intended procedure steps and a device failure may occur. It may be determined that the robotic system is not at fault, which may include fault logging and/or event logging. End of procedure auditing of autonomous operation may be logged based on procedure review, user reactions, and/or monitored outcomes to record success, failure, user choices, and/or impacts on subsequent steps. For example, automatic annotation of the recorded image with temporal overlay and device automation flags may be placed to provide context and situational awareness of the operation.

The control system (e.g., local facility and/or global main system) may be alerted of encountered issues. Intended use, indication of use and contraindications for device used during surgery may be provided. The medical devices may indicate within the IFU its intended use, indication of use and contraindications. During use of the devices (e.g., each device) the system may identify whether violations to the IFU were predicted to occur and may alert the user prior to the event. Provisions may be in place to override the system if surgeon deems it appropriate. For example, the control system may alert that the device is not to be used on ischemic tissue near the colon. The surgeon's determination may be that the area is the best place to proceed. The surgeon may override the system to continue. The event may be logged to be analyzed after the procedure. A number of firings for a device may be exceeded. It may be determined whether buttressing material was used for any of the firings which may impact total # of firings allowed. The procedure may have total procedure time constraints. For example, if the device has been set up for too long, the system may alert that elapsed time has been exceeded. The surgeon may override if decision to use is decided. Tissue may not be placed and oriented correctly in the jaws. Tissue placement and thickness may be determined. Insertion of a counterfeit cartridge may be determined. It may be ensured that the device is cleaned prior to cartridge reloading. The materials of the devices may be monitored to identify whether a patient has a hypersensitivity prior to use of the device and/or may alert the surgeon prior to the surgery. The issues may be aggregated into classifications. The classifications may include priority and/or criticality of failure, user confusion, and/or hardware failure (e.g., product inquiry). There may be escalation between classifications (e.g., frequency and/or severity). Low risk hardware issues may have redundant systems in place. If repeated use of redundant systems is initiated, it may use that as a trigger that the primary system is encountering issues. If the redundant system is only occasionally utilized, it may signify a necessary system or module reboot which may be considered standard operation

Self-service problem solving of the hub and attached systems may be provided. Automation of image search features may be used within a procedure database for solution options. Images captured from the scope may be bounced off the database to identify healthy vs diseased tissue as confirmation prior to device activation of energy and/or stapling to minimize leaks and/or complications. In examples, it may be used to identify an optimal next step based on issue to alert the surgeon. For example, if the surgeon completed a staple line and perfusion occurred, it may alert the surgeon of the best approach to address the leak based on tissue type known historical procedures. For example, the hub may provide a recommendation (e.g., in the form of an SNS message) that that suturing may be the best technique to address leak or that an energy modality that minimizes time may provide the best technique. Images may be confirmed to a database for navigation confirmation. For example, as the scope is navigating to the intended site, it may have confirmation/verification checks of images from the scope compared to a known database to confirm the surgeon is going in the correct direction and alert when off course. Network connection resolution may be provided. The magnitude of the failure may be automatically defined and an appropriate response to an experienced issue may be used to handle the issue relative to functional operation and needs on the system. For example, this may be used for failures in operating room displays. If a current operating room display is deemed defective during the procedure, the system may transfer the contents of display to alternative displays. In examples, it may split-up information. The system may place an order for detective components. The maintenance may call set-up for repair of faulty equipment. The system may alert issues for tracking. Low-risk hardware issues may have redundant systems in place. Major faults and minor faults and the automated responses by the hub may be provided. For example, the hub may experience an issue with the video output. The power supply to the system may fail causing the total loss of video feeds. This may be classified as a major fault condition. Overlay system failures may include software errors. To ensure that the correct input is selected, the hub system may accommodate various forms of output from visualization systems. Control measures may ensure that the correct input (e.g., one that has video running) is selected for output. The display of the overlay may be stopped if the main fails (e.g., it cannot be trusted.) In such a case, it may be executed on a softcore or hardcore redundant processor. Overlay system failures may include hardware failures. Redundant hardware may be used to maintain full functionality of both systems and may include a voting system (e.g., similar to full-redundant PCS). Redundant hardware may provide full video function without providing overlay function. For a switch over hardware solution, the system may have redundant power supplies. The solution being difficult to achieve may result in it being an active solution (e.g., too many standards to switch over).

Overlay system failures may include power supply failures. In examples, a visualization system may be provided. The visualization system may fault. For example, the video may be frozen (e.g., but otherwise correct looking), which may be associated with the highest risk. A fault may be no video, fuzzy or interrupted video, monochrome video, color changed video (e.g., lowest risk), etc. Video mitigation may be provided. For example, a control measure may be to provide sufficient visualization to safely remove the instruments from the patient, after which a different visual platform may be found. This control measure may include a device that may process the raw MIPI sensor output from the visualization system and may render a Bayer pattern on the monitor. For example, white light Bayer pattern sensors may output luminance values for pixels (e.g., pixel), line-by-line and frame-by-frame, regardless of the color of the pixel. A series of red, green and blue filters on individual pixels may filter the incoming white light to provide intensity values for those colors. The image signal processing may use the Bayer pattern signal to calculate the missing color data for the pixel location (e.g., each pixel location). This process may be called demosaicing. By taking the raw sensor signal, placing it in memory, reading it from memory at the timing required for an HDMI display, and outputting it to a display, a rudimentary monochrome image may be produced to allow safe removal of the instruments from the patient. Quickboot or archiving of operational use conditions just prior to the failure or restart may be used to restore operation to the previous state. System resource management may be provided.

1. A device for monitoring a first autonomous function associated with a surgical task, the device comprising:
a processor configured to:
perform the first autonomous function associated with the surgical task, wherein performing the first autonomous function is associated with control feedback;
monitor the first autonomous function by tracking one or more outputs associated with the control feedback; and
if the one or more outputs cross a failure threshold, switch from performing the first autonomous function to performing a second autonomous function associated with the surgical task, wherein the second autonomous function is associated with failure mitigation feedback.

2. The device of claim wherein the processor is further configured to:
if the one or more outputs cross the failure threshold, generate a failure magnitude based on the one or more outputs; and
adjust one or more parameters associated with the failure mitigation feedback based on the failure magnitude.

3. The device of claim 2, wherein the failure magnitude is generated based at least one of: surgical context, type of the first autonomous function, type of the second autonomous function, historical data associated with the first autonomous function, a number of outputs that crossed failure threshold, or a degree of the one or more outputs crossing the failure threshold.

4. The device of claim 1, wherein the processor is further configured to:
perform analysis on the tracked one or more outputs associated with the control feedback;
generate a comparison output based on the analysis; and
if the comparison output crosses the failure threshold, switch from performing the first autonomous function to performing the second autonomous function associated with the surgical task, wherein the second autonomous function is associated with the failure mitigation feedback.

5. The device of claim 4, wherein the processor is further configured to assign weight to each of the tracked one or more outputs based on at least one of: historical data, surgical context, type of the first autonomous function, type of the second autonomous function, historical data associated with the first autonomous function, a number of outputs that crossed failure threshold, or a degree of the one or more outputs crossing the failure threshold.

6. The device of claim 1, wherein the processor is farther configured to:
if the one or more outputs cross the failure threshold, send an indication of a failure message to a user of a surgical instrument associated with the surgical task, wherein the failure message comprises a failure type and a failure magnitude.

7. The device of 6, wherein the failure message comprises a set of recommendations based on type of the first autonomous function, type of the second autonomous function, historical data associated with the first autonomous function, a number of outputs that crossed failure threshold, or a degree of the one or more outputs crossing the failure threshold.

8. The device of claim 1, wherein the processor is further configured to determine the failure threshold and failure mitigation feedback based on training data.

9. The device of claim 8, wherein the training data is based on at least one of: historical data, surgical context, type of the first autonomous function, type of the second autonomous function, historical data associated with the first autonomous function, a number of outputs that crossed failure threshold, or a degree of the one or more outputs crossing the failure threshold.

10. The device of claim 1, wherein the failure threshold is adjusted based on situational awareness.

11. A method for monitoring, a first autonomous function associated with a surgical task, the method comprising:
performing the first autonomous function associated with the surgical task, wherein performing the first autonomous function is associated with control feedback;
monitoring the first autonomous function by tracking one or more outputs associated with the control feedback; and
if the one or more outputs cross a failure threshold, switching from performing the first autonomous function to performing a second autonomous function associated with the surgical task, wherein the second autonomous function is associated with failure mitigation feedback.

12. The method of claim 11, further comprising:
if the one or more outputs cross the failure threshold, generating a failure magnitude based on the one or more outputs; and
adjusting one or more parameters associated with the failure mitigation feedback based on the failure magnitude.

13. The method of claim 12, wherein the failure magnitude is generated based at least one of: surgical context, type of the first autonomous function, type of the second autonomous function, historical data associated with the first autonomous function, a number of outputs that crossed failure threshold, or a degree of the one or more outputs crossing failure threshold.

14. The method of claim 11, further comprising:
performing analysis on the tracked one or more outputs associated with the control feedback;
generating a comparison output based on the analysis; and
if the comparison output crosses the failure threshold, switching from performing the first autonomous function to performing the second autonomous function associated with the surgical task, wherein the second autonomous function is associated with the failure mitigation feedback.

15. The method of claim 14, further comprising:
assigning weight to each of the tracked one or more outputs based on at least one of: historical data, surgical context, type of the first autonomous function, type of the second autonomous function, historical data associated with the first autonomous function, a number of outputs that crossed failure threshold, or a degree of the one or more outputs crossing the failure threshold.

16. The method of claim 11, further comprising:
the one Of more outputs cross the failure threshold, sending an indication of a failure message to a user of a surgical instrument associated with the surgical task, wherein the failure message comprises a failure type and a failure magnitude.

17. The method of 16, wherein the failure message comprises a set of recommendations based on type of the first autonomous function, type of the second autonomous function, historical data associated with the first autonomous function, a number of outputs that crossed failure threshold, or a degree of the one or more outputs crossing the failure threshold.

18. The method of claim 11, further comprising:
determining the failure threshold and failure mitigation feedback based on training data.

19. The method of claim 18, wherein the training data is based on at least one of: historical data, surgical context, type of the first autonomous function, type of the second autonomous function, historical data associated with the first autonomous function, a number of outputs that crossed failure threshold, or a degree of the one or more outputs crossing the failure threshold.

20. The method of claim 11, wherein the failure threshold is adjusted based on situational awareness.
